(19) United States
(12) Patent Application Publication
(10) Pub. No.: US 2014/0227176 A1
(43) Pub. Date: Aug. 14, 2014

(54) Radioactive and/or Magnetic Metal Nanoparticles and Process and Apparatus for Synthesizing Same

(71) Applicant: Universite Laval, Quebec (CA)

(72) Inventors: Fortin Marc-Andre, Quebec (CA); Christian Sarra-Bournet, Quebec (CA); Mathieu Letourneau, Quebec (CA); Gaetan Laroche, Quebec (CA)

(21) Appl. No.: 13/996,333

(22) PCT Filed: Dec. 20, 2011
(86) PCT No.: PCT/CA2011/001423

§ 371 Date: Sep. 16, 2013

Related U.S. Application Data
(60) Provisional application No. 61/424,868, filed on Dec. 20, 2010.

(51) Int. Cl. A61K 51/12; G01N 33/60; C01G 49/02; C01G 45/02; C01F 17/00

(52) U.S. Cl. CPC A61K 51/1244; C01G 45/02; C01F 17/0043; C01G 49/02; G01N 33/60; C01P 2006/42; C01P 2006/44; A61K 2201/103
USPC 424/1.29; 423/605; 423/263; 423/632; 423/249; 422/186.04; 422/159; 204/164; 435/29

(57) ABSTRACT

A process for manufacturing magnetic and/or radioactive metal nanoparticles, the process comprising: preparing an electrolyte solution including metal ions and a stabilizer; generating a plasma at an interface of the electrolyte solution at atmospheric pressure; and recovering magnetic and/or radioactive metal nanoparticles. The magnetic metal nanoparticles can comprise magnetoradioactive nanoparticles. The magnetic metal nanoparticles can be used as MRI contrast agents and the magnetoradioactive nanoparticles can also be used as contrast agents and for dual PET/MRI applications. It also relates to a multi-plasma apparatus for synthesizing nanoparticles.

### CROSS-REFERENCE TO RELATED APPLICATIONS

This application claims priority under 35 USC §119(e) of U.S. provisional patent application 61/424,868 filed on Dec. 20, 2010, the specification of which is hereby incorporated by reference. This application is a national phase entry of PCT patent application serial number PCT/CA2011/001423 filed on Dec. 20, 2011, now pending, designating the United States of America.

### FIELD OF THE INVENTION

The technical field relates to radioactive and/or magnetic metal nanoparticles and to processes for synthesizing same. It also relates to a process and an apparatus to synthesize the nanoparticles.

### BACKGROUND OF THE INVENTION

Microscale plasmas (or microplasmas) are confined within sub-millimeter cavities in at least one dimension. They provide high pressure stability, non-equilibrium thermodynamics, non-Maxwellian electron energy distribution functions, high electron densities, excimer generation, and new plasma sheath dynamics. The principal advantage of microplasmas in the context of materials science, is the generation of stable plasmas in atmospheric pressure conditions (see for instance, Tachibana et al, Current Status of Microplasma Research, in IEEE Transactions on Electrical and Electronic Engineering 2006, 1, 145; D. Mariotti, et al., Journal of Physics D-Applied Physics 2010, 43, 323001-323021; R. Foest, et al., International Journal of Mass Spectrometry 2006, 248, 87-102; R. M. Sankaran, et al., Journal of Applied Physics 2002, 92, 2406-2411; R. M. Sankaran, et al., Journal of Physics D-Applied Physics 2003, 36, 2914-2921; K. H. Becker, et al., Journal of Physics D-Applied Physics 2006, 39, R55-R70; Kogelschatz, Applications of Microplasmas and Microreactor Technology, Contributions to Plasma Physics 2007, nos 1-2, 80-88.). Due to equipment and reactor maintenance, high costs are associated with the generation of low-pressure plasmas. However, operation of plasmas near or at atmospheric pressure is cheaper, easier to implement and more appropriate for continuous industrial processes. Microplasmas can be ignited in hollow cathodes, a geometry that facilitates ionization through the oscillatory motion of electrons (K. H. Schoenbach, et al., Applied Physics Letters 1996, 68, 13-15). Other geometry can also be used, such as a microplasma jet or dielectric barrier discharge (DBD) configuration (K. Tachibana, IEEJ Transaction on Electrical and Electronic Engineering 2006, 1, 145). Then, such plasmas can be coupled to liquids to grow materials in solution, through the initiation of electrochemical reactions such as anodic dissolution of solid electrodes or reduction of dispersed metal salts. (W. H. Chiang, et al., Plasma Sources Science & Technology 2010, 19; C. Richmonds, et al., Applied Physics Letters 2008, 93). Free electrons and ions expelled from microplasma sources, and projected at the surface of liquids containing metal ions, initiate a current flow inside the electrolyte. By introducing in the electrolyte an anode biased against the hollow plasma source (the cathode), a current flow is channeled and rapid and efficient electrochemical reactions take place at the plasma-liquid interface. In certain conditions, metal nanoparticles are generated in the liquid facing the cathode, and this process is one of the most rapid of all metal nanoparticle synthesis procedures. The process has been recently demonstrated for gold and silver nanoparticles (W. H. Chiang, et al., Plasma Sources Science & Technology 2010, 19; C. Richmonds, et al., Applied Physics Letters 2008, 93); the fine and narrow particle size distributions achieved thereby, consisted of high purity products dispersed in aqueous media. Using this procedure, the colloidal particles are neither contaminated by adherence to a deposition substrate, nor by the use of chemical reducing agents.

US patent application no. 2011/0048960 discloses an electrochemical cell including a container at atmospheric pressure. The container comprises a liquid electrolyte including noble metal particles and a first electrode at least partially immersed in the electrolyte. A plasma source is spaced apart from a surface of the electrolyte by a predetermined spacing, and a plasma spans the predetermined spacing to contact the surface of the electrolyte. Electrochemical reactions involving the second electrode are initiated in the electrolyte. More particularly, the metal ions in the electrolyte are reduced by free electrons from the plasma to form noble metal nanoparticles in the electrolyte such as Ag, Au, and Pt nanoparticles.

Nanoparticles made of magnetic elements such as Fe, Mn, Gd, Ni, Cr, Cu, and Co are used in various applications such as catalytic processes and biomedical therapy and imaging. (J. Park, et al., Nature Materials 2004, 3, 891-895; J. Y. Park, et al., ACS Nano 2009, 3, 3663-3669; L. Josephson, et al., Magn Reson Imaging 1988, 6, 647-53; M. Engström, et al., Magma 2006, 19, 180-6; H. B. Na, et al., Advanced Materials 2009, 21, 2133-2148; C. Corot, et al., Adv Drug Deliv Rev 2006, 58, 1471-504). One of the main current limitations of these products is the time required to synthesize particles of fine and narrow distributions, not contaminated by chemical reducing agents, purified and suspended in different aqueous media. Conventional procedures used to synthesize ultra-fine nanoparticles made of the elements Fe, Mn, Gd, Ni, Cr, Cu, and Co, require colloidal synthesis techniques, which almost invariably call for the expert touch of a skilled chemist in order to achieve optimal nanoparticle properties. A majority of ultra-fine nanoparticle synthesis procedures are performed in organic solvents which produce particles that are covered with hydrophobic molecules such as oleic acid; the transfer of those particles to an aqueous suspension, such as cell culture medium, blood or physiological saline, first requires an appropriate ligand exchange procedure, followed by an adequate purification step. Each one of these procedures adds time and tedious manipulations to the overall procedure and taken together, limit the potential for widespread production and utilization of the particles.

Furthermore, both nuclear imaging modalities and internal radiotherapeutical approaches require the use of radioactive tracers, or nanoparticles, and the radionuclide half-life of these products can be as short as a few hours only.

There is thus a need for a process and an apparatus to rapidly and efficiently synthesize metal radioactive and/or magnetic metal nanoparticles by an atmospheric plasma synthesis route.

### SUMMARY OF THE INVENTION

It is therefore an aim of the present invention to address the above mentioned issues.

According to a general aspect, there is provided a process for synthesizing magnetic metal nanoparticles, the process comprising: preparing an electrolyte solution including metal ions and a surfactant; generating a plasma at an interface of the electrolyte solution at atmospheric pressure; and recovering the synthesized magnetic metal nanoparticles.

According to another general aspect, there is provided magnetoradioactive nanoparticles synthesized by a plasma-liquid electrochemical process at atmospheric pressure and comprising a magnetic metallic element content and a radioactive atom content.

According to still another general aspect, there is provided a process for manufacturing magnetoradioactive nanoparticles, the process comprising: preparing an electrolyte solution including metal ions and a surfactant; adding radioactive atoms to the electrolyte solution; generating a plasma at an interface of the electrolyte solution and air at atmospheric pressure; and recovering the synthesized magnetoradioactive metal nanoparticles.

According to a further general aspect, there is provided a process for synthesizing magnetic metal nanoparticles, the process comprising: preparing an electrolyte solution including metal ions capable of forming magnetic metal nanoparticles and a stabilizer; generating, at atmospheric pressure, at least one plasma directed towards an interface of the electrolyte solution; and recovering, from the electrolyte solution, the synthesized magnetic metal nanoparticles.

In one embodiment, the metal ions comprise at least one of a ferromagnetic metal and a paramagnetic metal. The at least one of the ferromagnetic metal and the paramagnetic metal can comprise at least one of Fe, Co, Ni, Mn, Cr, Gd, Cu, Eu, and Dy.

In one embodiment, the synthesized magnetic metal nanoparticles comprise at least one of magnetic metal oxide nanoparticles, magnetic metal phosphate nanoparticles, magnetic metal hydroxide nanoparticles, and mixtures thereof. For instance, the synthesized magnetic metal nanoparticles can comprise magnetic metallic nanoparticles selected from the group consisting of: Fe, Cu, and mixtures thereof. For instance, the synthesized magnetic metal nanoparticles can comprise magnetic metal oxide nanoparticles with a metal selected from the group consisting of: Gd, Mn, Fe, Dy, Co, Cr, Ni, Ru, Rh, Pd, Or, Ir, Pt, Pb, Sn, Ge, B, Al, Ga, In, Tl, Li, Na, K, Rb, Cs, Fr, Be, Mg, Ca, Sr, Ba, Ra, Cu, Cd, Zn, Sc, Y, La, Ce, Pr, Nd, Sm, Eu, Tb, Ho, Er, Tm, Yb, Lu, and Pm. For instance, the magnetic metal nanoparticles can comprise magnetic metal phosphate nanoparticles with a metal selected from the group consisting of: Gd, Mn, Fe, Dy, Co, Cr, Ni, Ru, Rh, Pd, Or, Ir, Pt, Pb, Sn, Ge, B, Al, Ga, In, Tl, Li, Na, K, Rb, Cs, Fr, Be, Mg, Ca, Sr, Ba, Ra, Cu, Cd, Zn, Sc, Y, La, Ce, Pr, Nd, Sm, Eu, Tb, Ho, Er, Tm, Yb, Lu, and Pm. For instance, the magnetic metal nanoparticles can comprise magnetic metal hydroxide nanoparticles with a metal selected from the group consisting of: Gd, Mn, Fe, Dy, Co, Cr, Ni, Ru, Rh, Pd, Or, Ir, Pt, Pb, Sn, Ge, B, Al, Ga, In, Tl, Li, Na, K, Rb, Cs, Fr, Be, Mg, Ca, Sr, Ba, Ra, Cu, Cd, Zn, Sc, Y, La, Ce, Pr, Nd, Sm, Eu, Tb, Ho, Er, Tm, Yb, Lu, and Pm.

In one embodiment, the generating step further comprises generating simultaneously a plurality of plasma directed towards the interface of the electrolyte solution

In one embodiment, the electrolyte solution is an aqueous electrolyte solution obtained by dissolving a metal ion precursor and the stabilizer in pure water. The metal ion precursor can be selected from the group consisting of: metal chlorides, metal nitrates, metal acetates, organometallics, and mixtures thereof. The metal ion precursor can be selected from the group consisting of: MnCl₂*4H₂O, Gd(NO₃)₃*6H₂O, GdCl₃*6H₂O, FeCl₃, MnCl₂, Gd(NO₃)₃, GdCl₃, FeCl₃, and mixtures thereof.

In one embodiment, the step of generating the at least one plasma is carried out in ambient air.

In one embodiment, the stabilizer comprises a surfactant selected from the group consisting of: fructose, dextran, polyethylene glycol, dimercaptosucccinic acid (DMSA), citric acid, and mixtures thereof.

In one embodiment, the electrolyte solution comprises between 0.01 mM and 1 M of the stabilizer and between 0.01 mM and 1 M of the metal ions.

In one embodiment, the stabilizer is biocompatible and/or comprises —OH containing molecules.

In one embodiment, the synthesized magnetic metal nanoparticles are covered by the stabilizer in the electrolyte solution.

In one embodiment, the generating step comprises injecting at least one of an inert gas and a reactive gas towards the electrolyte solution in at least one gas channel defined in at least one cathode and creating a potential difference between the at least one cathode and at least one anode. The at least one of the inert gas and the reactive gas can be selected from the group consisting of: argon, helium, H_{z}, N₂, NH₃, and mixtures thereof.

In one embodiment, the synthesized magnetic metal nanoparticles are smaller than about 100 nanometers. In another embodiment, the synthesized magnetic metal nanoparticles are between about 1 nm and about 50 nm. In still another embodiment, the synthesized magnetic metal nanoparticles are between about 1 nm and about 15 nm.

In one embodiment, the process further comprises controlling a temperature of the electrolyte solution between about 0° C. and about 50° C.; and/or controlling a pH of the electrolyte solution between about 5 and about 7; and/or adding at least one —OH containing solution to the electrolyte solution during the generating step.

In one embodiment, the preparing step further comprises adding at least one radioactive atom to the electrolyte solution.

In one embodiment, the generating step further comprises generating at least one micro-plasma.

In one embodiment, there is provided magnetic metal nanoparticles obtained by the above-described process. The magnetic metal nanoparticles can be used as MRI contrast agent. The magnetic metal nanoparticles containing at least one iron oxide and obtained by the above-described process can be used as magnetotherapeutic agent or cell labeling agent.

According to another general aspect, there is provided a process for synthesizing radioactive metal nanoparticles, the process comprising: preparing an electrolyte solution including metal ions, a stabilizer, and at least one radioactive atom; generating, at atmospheric pressure, at least one plasma directed towards an interface of the electrolyte solution; and recovering, from the electrolyte solution, the synthesized radioactive metal nanoparticles with the at least one radioactive atom integrated therein.

In one embodiment, the at least one radioactive atom is added as at least one of a radioactive salt, a radioactive chloride, a radioactive nitrate, a radioactive acetate, and a radioactive organometallic.

In one embodiment, the at least one radioactive atom is at least one of a positron emitter, a beta emitter, an alpha emitter, and a gamma emitter.

In one embodiment, the synthesized radioactive metal nanoparticles comprise a core including a crystal containing the at least one radioactive atom.

In one embodiment, the at least one radioactive atom comprises at least one radioisotope.

In one embodiment, a concentration of the at least one radioactive atom in the synthesized radioactive metal nanoparticles ranges between about 0.01 and about 5.0 mol-%.

In one embodiment, the electrolyte solution comprises between about 0.001 and about 50 mCi of the at least one radioactive atom per mL of the electrolyte solution

In one embodiment, the at least one radioactive atom is in a concentration below about 1 wt % in the synthesized radioactive metal nanoparticles.

In one embodiment, the at least one radioactive atom is selected from the group consisting of: ¹¹C, ¹³N, ¹⁵O, ^{66/8}Ga, ⁶⁰Cu, ⁵²Fe, ^{61/2/4}Cu, ^{62/3}Zn, ^{70/1/4}As, ^{75/6}Br, ⁸²Rb, ⁸⁶Y, ⁸⁹Zr, ¹¹⁰In, ^{120/4}I, ¹²²Xe, ¹⁸F ^{99m}Tc, ^{123/5/131}I, ⁶⁷Ga, ¹¹¹In, ²⁰¹Tl, ³²P, ³⁹Sr, ¹⁶⁵Dy, ¹⁰⁹Pd, ¹⁶⁹Er, ¹⁰⁵Rh, ⁷⁷As, ¹⁴⁹Pm, ^{153/7}Sm, ¹⁷⁷Lu, ^{212/3}Bi, ^{186/8}Re, ⁶⁷Cu, ⁹⁰Y, ^{115m}In, ²¹¹At, ¹⁶⁶Ho, ¹⁹⁹Au, ¹¹¹Ag, ⁴⁷Sc, ^{193m}Pt, ^{117m}Sn, and mixtures thereof.

In one embodiment, the synthesized radioactive metal nanoparticles comprise at least one of metal oxide nanoparticles, metal phosphate nanoparticles, magnetic metal hydroxide nanoparticles, and mixtures thereof.

In one embodiment, the synthesized radioactive metal nanoparticles comprise metallic nanoparticles selected from the group consisting of: Fe, Cu, and mixtures thereof. The synthesized radioactive metal nanoparticles can comprise metal oxide nanoparticles with a metal selected from the group consisting of: Gd, Mn, Fe, Dy, Co, Cr, Ni, Ru, Rh, Pd, Or, Ir, Pt, Pb, Sn, Ge, B, Al, Ga, In, Tl, Li, Na, K, Rb, Cs, Fr, Be, Mg, Ca, Sr, Ba, Ra, Cu, Cd, Zn, Sc, Y, La, Ce, Pr, Nd, Sm, Eu, Tb, Ho, Er, Tm, Yb, Lu, and Pm. The synthesized radioactive metal nanoparticles can comprise metal phosphate nanoparticles with a metal selected from the group consisting of: Gd, Mn, Fe, Dy, Co, Cr, Ni, Ru, Rh, Pd, Or, Ir, Pt, Pb, Sn, Ge, B, Al, Ga, In, Tl, Li, Na, K, Rb, Cs, Fr, Be, Mg, Ca, Sr, Ba, Ra, Cu, Cd, Zn, Sc, Y, La, Ce, Pr, Nd, Sm, Eu, Tb, Ho, Er, Tm, Yb, Lu, and Pm. The synthesized radioactive metal nanoparticles can comprise metal hydroxide nanoparticles with a metal selected from the group consisting of: Gd, Mn, Fe, Dy, Co, Cr, Ni, Ru, Rh, Pd, Or, Ir, Pt, Pb, Sn, Ge, B, Al, Ga, In, Tl, Li, Na, K, Rb, Cs, Fr, Be, Mg, Ca, Sr, Ba, Ra, Cu, Cd, Zn, Sc, Y, La, Ce, Pr, Nd, Sm, Eu, Tb, Ho, Er, Tm, Yb, Lu, and Pm.

In one embodiment, the generating step further comprises generating simultaneously a plurality of plasma directed towards the interface of the electrolyte solution.

In one embodiment, the electrolyte solution is an aqueous electrolyte solution obtained by dissolving a metal ion precursor and the stabilizer in pure water. The metal ion precursor can be selected from the group consisting of: metal chlorides, metal nitrates, metal acetates, organometallics, and mixtures thereof. The metal ion precursor can be selected from the group consisting of: MnCl₂*4H₂O, Gd(NO₃)₃*6H₂O, GdCl₃*6H₂O, FeCl₃, MnCl₂, Gd(NO₃)₃, GdCl₃, FeCl₃, and mixtures thereof.

In one embodiment, the step of generating the at least one plasma is carried out in ambient air.

In one embodiment, the stabilizer comprises a surfactant selected from the group consisting of: fructose, dextran, polyethylene glycol, dimercaptosucccinic acid (DMSA), citric acid, and mixtures thereof.

In one embodiment, the electrolyte solution comprises between 0.01 mM and 1 M of the stabilizer.

In one embodiment, the stabilizer is biocompatible and/or comprises —OH containing molecules.

In one embodiment, the synthesized radioactive metal nanoparticles are covered by the stabilizer in the electrolyte solution.

In one embodiment, the generating step comprises injecting at least one of an inert gas and a reactive gas towards the electrolyte solution in at least one gas channel defined in at least one cathode and creating a potential difference between the at least one cathode and at least one anode. The at least one of the inert gas and the reactive gas can be selected from the group consisting of: argon, helium, H₂, N₂, NH₃, and mixtures thereof.

In one embodiment, the synthesized radioactive metal nanoparticles are smaller than about 100 nanometers. In another embodiment, the synthesized radioactive metal nanoparticles are between about 1 nm and about 50 nm. In still another embodiment, the synthesized radioactive metal nanoparticles are between about 1 nm and about 15 nm.

In one embodiment, the process further comprises controlling a temperature of the electrolyte solution between about 0° C. and about 50° C.; and/or controlling a pH of the electrolyte solution between about 5 and about 7; and/or adding at least one —OH containing solution to the electrolyte solution during the generating step.

In one embodiment, the generating step further comprises generating at least one micro-plasma.

In one embodiment, there is provided radioactive metal nanoparticles obtained by the process described above. The radioactive metal nanoparticles, obtained by the process described above, can be comprised in a radiotherapeutic agent or a radioactive tracer for nuclear image procedures.

According to still another general aspect, there is provided a process for synthesizing magnetoradioactive metal nanoparticles, the process comprising: preparing an electrolyte solution including metal ions capable of forming magnetic metal nanoparticles, a stabilizer, and at least one radioactive atom; generating, at atmospheric pressure, at least one plasma directed towards an interface of the electrolyte solution; and recovering, from the electrolyte solution, the synthesized magnetoradioactive metal nanoparticles.

In one embodiment, the metal ions comprise at least one of a ferromagnetic metal and a paramagnetic metal. The at least one of the ferromagnetic metal and the paramagnetic metal can comprise at least one of Fe, Co, Ni, Mn, Cr, Cu, Gd, Eu, and Dy.

In one embodiment, the synthesized magnetoradioactive metal nanoparticles comprise at least one of magnetic metal oxide nanoparticles, magnetic metal phosphate nanoparticles, magnetic metal hydroxide nanoparticles, and mixtures thereof.

In one embodiment, the synthesized magnetoradioactive metal nanoparticles comprise magnetic metallic nanoparticles selected from the group consisting of: Fe, Cu, and mixtures thereof. The synthesized magnetoradioactive metal nanoparticles can comprise magnetic metal oxide nanoparticles with a metal selected from the group consisting of: Gd, Mn, Fe, Dy, Co, Cr, Ni, Ru, Rh, Pd, Or, Ir, Pt, Pb, Sn, Ge, B, Al, Ga, In, Tl, Li, Na, K, Rb, Cs, Fr, Be, Mg, Ca, Sr, Ba, Ra, Cu, Cd, Zn, Sc, Y, La, Ce, Pr, Nd, Sm, Eu, Tb, Ho, Er, Tm, Yb, Lu, and Pm. The synthesized magnetoradioactive metal nanoparticles can comprise magnetic metal phosphate nanoparticles with a metal selected from the group consisting of: Gd, Mn, Fe, Dy, Co, Cr, Ni, Ru, Rh, Pd, Or, Ir, Pt, Pb, Sn, Ge, B, Al, Ga, In, Tl, Li, Na, K, Rb, Cs, Fr, Be, Mg, Ca, Sr, Ba, Ra, Cu, Cd, Zn, Sc, Y, La, Ce, Pr, Nd, Sm, Eu, Tb, Ho, Er, Tm, Yb, Lu, and Pm. The synthesized magnetoradioactive metal nanoparticles can comprise magnetic metal hydroxide nanoparticles with a metal selected from the group consisting of: Gd, Mn, Fe, Dy, Co, Cr, Ni, Ru, Rh, Pd, Or, Ir, Pt, Pb, Sn, Ge, B, Al, Ga, In, Tl, Li, Na, K, Rb, Cs, Fr, Be, Mg, Ca, Sr, Ba, Ra, Cu, Cd, Zn, Sc, Y, La, Ce, Pr, Nd, Sm, Eu, Tb, Ho, Er, Tm, Yb, Lu, and Pm.

In one embodiment, the at least one radioactive atom is added as at least one of a radioactive salt, a radioactive chloride, a radioactive nitrate, a radioactive acetate, and a radioactive organometallic.

In one embodiment, the at least one radioactive atom is at least one of a positron emitter, a beta emitter, an alpha emitter, and a gamma emitter.

In one embodiment, the synthesized radioactive metal nanoparticles comprise the at least one radioactive atom integrated to the synthesized radioactive metal nanoparticles.

In one embodiment, the synthesized radioactive metal nanoparticles comprise a core including a crystal containing the at least one radioactive atom.

In one embodiment, the at least one radioactive atom comprises at least one radioisotope.

In one embodiment, a concentration of the at least one radioactive atom in the synthesized radioactive metal nanoparticles ranges between about 0.01 and about 5.0 mol-%.

In one embodiment, the electrolyte solution comprises between about 0.001 and about 50 mCi of the at least one radioactive atom per mL of the electrolyte solution.

In one embodiment, the at least one radioactive atom is in a concentration below about 1 wt % in the synthesized radioactive metal nanoparticles.

In one embodiment, the at least one radioactive atom is selected from the group consisting of: ¹¹C, ¹³N, ¹⁵O, ^{66/8}Ga, ⁶⁰Cu, ⁵²Fe, ^{61/2/4}Cu, ^{62/3}Zn, ^{70/1/4}As, ^{75/6}Br, ⁸²Rb, ⁸⁶Y, ⁸⁹Zr, ¹¹⁰In, ^{120/4}I, ¹²²Xe, ¹⁸F ^{99m}Tc, ^{123/5/131}I, ⁶⁷Ga, ¹¹¹In, ²⁰¹Tl, ³²P, ³⁹Sr, ¹⁶⁵Dy, ¹⁰⁹Pd, ¹⁶⁹Er, ¹⁰⁵Rh, ⁷⁷As, ¹⁴⁹Pm, ^{153/7}Sm, ¹⁷⁷Lu, ^{212/3}Bi, ^{186/8}Re, ⁶⁷Cu, ⁹⁰Y, ^{115m}In, ²¹¹At, ¹⁶⁶Ho, ¹⁹⁹Au, ¹¹¹Ag, ⁴⁷Sc, ^{193m}Pt, ^{117m}Sn, and mixtures thereof.

In one embodiment, the generating step further comprises generating simultaneously a plurality of plasma directed towards the interface of the electrolyte solution.

In one embodiment, the electrolyte solution is an aqueous electrolyte solution obtained by dissolving a metal ion precursor and the stabilizer in pure water. The metal ion precursor can be selected from the group consisting of: metal chlorides, metal nitrates, metal acetates, organometallics, and mixtures thereof. The metal ion precursor can be selected from the group consisting of: MnCl₂*4H₂O, Gd(NO₃)₃*6H₂O, GdCl₃*6H₂O, FeCl₃, MnCl₂, Gd(NO₃)₃, GdCl₃, FeCl₃, and mixtures thereof.

In one embodiment, the step of generating the at least one plasma is carried out in ambient air.

In one embodiment, the stabilizer comprises a surfactant selected from the group consisting of: fructose, dextran, polyethylene glycol, dimercaptosucccinic acid (DMSA), citric acid, and mixtures thereof.

In one embodiment, the electrolyte solution comprises between 0.01 mM and 1 M of the stabilizer and between 0.01 mM and 1 M of the metal ions.

In one embodiment, the stabilizer is biocompatible and/or comprises —OH containing molecules.

In one embodiment, the synthesized magnetoradioactive metal nanoparticles are covered by the stabilizer in the electrolyte solution.

In one embodiment, the generating step comprises injecting at least one of an inert gas and a reactive gas towards the electrolyte solution in at least one gas channel defined in at least one cathode and creating a potential difference between the at least one cathode and at least one anode. The at least one of the inert gas and the reactive gas can be selected from the group consisting of: argon, helium, H₂, N₂, NH₃, and mixtures thereof.

In one embodiment, the synthesized magnetoradioactive metal nanoparticles are smaller than about 100 nanometers. In another embodiment, the synthesized magnetoradioactive metal nanoparticles are between about 1 nm and about 50 nm. In still another embodiment, the synthesized magnetoradioactive metal nanoparticles are between about 1 nm and about 15 nm.

In one embodiment, the process further comprises controlling a temperature of the electrolyte solution between about 0° C. and about 50° C.; and/or controlling a pH of the electrolyte solution between about 5 and about 7; and/or adding at least one —OH containing solution to the electrolyte solution during the generating step.

In one embodiment, the generating step further comprises generating at least one micro-plasma

In one embodiment, there is provided magnetoradioactive metal nanoparticles obtained by the process described above. The magnetoradioactive metal nanoparticles can be included in a PET tracer, a contrast agent, a MRI tracer, a cell labeling agent, or a magnetotherapeutic agent. They can also be used for dual PET/MRI.

According to still another general aspect, there is provided radioactive metal nanoparticles synthesized by a plasma-liquid electrochemical process at atmospheric pressure and comprising a metal element content and at least one radioactive atom integrated in a crystal of the synthesized radioactive metal nanoparticles.

In one embodiment, the synthesized radioactive metal nanoparticles comprise at least one of magnetic metal oxide nanoparticles, magnetic metal phosphate nanoparticles, magnetic metal hydroxide nanoparticles, and mixtures thereof. The synthesized radioactive metal nanoparticles can comprise metal oxide nanoparticles with a metal selected from the group consisting of: Gd, Mn, Fe, Dy, Co, Cr, Ni, Ru, Rh, Pd, Or, Ir, Pt, Pb, Sn, Ge, B, Al, Ga, In, Tl, Li, Na, K, Rb, Cs, Fr, Be, Mg, Ca, Sr, Ba, Ra, Cu, Cd, Zn, Sc, Y, La, Ce, Pr, Nd, Sm, Eu, Tb, Ho, Er, Tm, Yb, Lu, and Pm. The synthesized radioactive metal nanoparticles can comprise magnetic metal phosphate nanoparticles with a metal selected from the group consisting of: Gd, Mn, Fe, Dy, Co, Cr, Ni, Ru, Rh, Pd, Or, Ir, Pt, Pb, Sn, Ge, B, Al, Ga, In, Tl, Li, Na, K, Rb, Cs, Fr, Be, Mg, Ca, Sr, Ba, Ra, Cu, Cd, Zn, Sc, Y, La, Ce, Pr, Nd, Sm, Eu, Tb, Ho, Er, Tm, Yb, Lu, and Pm. The synthesized radioactive metal nanoparticles can comprise magnetic metal hydroxide nanoparticles with a metal selected from the group consisting of: Gd, Mn, Fe, Dy, Co, Cr, Ni, Ru, Rh, Pd, Or, Ir, Pt, Pb, Sn, Ge, B, Al, Ga, In, Tl, Li, Na, K, Rb, Cs, Fr, Be, Mg, Ca, Sr, Ba, Ra, Cu, Cd, Zn, Sc, Y, La, Ce, Pr, Nd, Sm, Eu, Tb, Ho, Er, Tm, Yb, Lu, and Pm.

In one embodiment, the at least one radioactive atom comprises at least one of a positron emitter, a beta emitter, an alpha emitter, and a gamma emitter.

In one embodiment, the radioactive metal nanoparticles further comprise a core including the crystal containing the at least one radioactive atom.

In one embodiment, a concentration of the at least one radioactive atom in the synthesized radioactive metal nanoparticles ranges between about 0.01 and about 5.0 mol-%.

In one embodiment, the at least one radioactive atom is in a concentration below about 1 wt % in the synthesized radioactive metal nanoparticles.

In one embodiment, the at least one radioactive atom is selected from the group consisting of: ¹¹C, ¹³N, ¹⁵O, ^{66/8}Ga, ⁶⁰Cu, ⁵²Fe, ^{61/2/4}Cu, ^{62/3}Zn, ^{70/1/4}As, ^{75/6}Br, ⁸²Rb, ⁸⁶Y, ⁸⁹Zr, ¹¹⁰In, ^{120/4}I, ¹²²Xe, ¹⁸F ^{99m}Tc, ^{123/5/131}I, ⁶⁷Ga, ¹¹¹In, ²⁰¹Tl, ³²P, ³⁹Sr, ¹⁶⁵Dy, ¹⁰⁹Pd, ¹⁶⁹Er, ¹⁰⁵Rh, ⁷⁷As, ¹⁴⁹Pm, ^{153/7}Sm, ¹⁷⁷Lu, ^{212/3}Bi, ^{186/8}Re, ⁶⁷Cu, ⁹⁰Y, ^{115m}In, ²¹¹At, ¹⁶⁶Ho, ¹⁹⁹Au, ¹¹¹Ag, ⁴⁷Sc, ^{193m}Pt, ^{117m}Sn, and mixtures thereof.

In one embodiment, the synthesized radioactive metal nanoparticles are covered by a stabilizer. The stabilizer can comprise a surfactant selected from the group consisting of: fructose, dextran, polyethylene glycol, dimercaptosucccinic acid (DMSA), citric acid, and mixtures thereof. The stabilizer can be biocompatible.

In one embodiment, the synthesized radioactive metal nanoparticles are smaller than about 100 nanometers. In another embodiment, the synthesized radioactive metal nanoparticles are between about 1 nm and about 50 nm. In still another embodiment, the synthesized radioactive metal nanoparticles are between about 1 nm and about 15 nm.

According to a further general aspect, there is provided magnetoradioactive metal nanoparticles synthesized by a plasma-liquid electrochemical process at atmospheric pressure and comprising a metal element content and at least one radioactive atom integrated in a crystal of the synthesized magnetoradioactive metal nanoparticles.

In one embodiment, the metal element content comprises at least one of a ferromagnetic metal and a paramagnetic metal. The at least one of the ferromagnetic metal and the paramagnetic metal can comprise at least one of Fe, Co, Ni, Mn, Cr, Cu, Eu, Gd, and Dy.

In one embodiment, the synthesized magnetoradioactive metal nanoparticles comprise at least one of magnetic metal oxide nanoparticles, magnetic metal phosphate nanoparticles, magnetic metal hydroxide nanoparticles, and mixtures thereof.

In one embodiment, the synthesized magnetoradioactive metal nanoparticles comprise magnetic metallic nanoparticles selected from the group consisting of: Fe, Cu, and mixtures thereof. The synthesized magnetoradioactive metal nanoparticles can comprise magnetic metal oxide nanoparticles with a metal selected from the group consisting of: Gd, Mn, Fe, Dy, Co, Cr, Ni, Ru, Rh, Pd, Or, Ir, Pt, Pb, Sn, Ge, B, Al, Ga, In, Tl, Li, Na, K, Rb, Cs, Fr, Be, Mg, Ca, Sr, Ba, Ra, Cu, Cd, Zn, Sc, Y, La, Ce, Pr, Nd, Sm, Eu, Tb, Ho, Er, Tm, Yb, Lu, and Pm. The synthesized magnetoradioactive metal nanoparticles can comprise magnetic metal phosphate nanoparticles with a metal selected from the group consisting of: Gd, Mn, Fe, Dy, Co, Cr, Ni, Ru, Rh, Pd, Or, Ir, Pt, Pb, Sn, Ge, B, Al, Ga, In, Tl, Li, Na, K, Rb, Cs, Fr, Be, Mg, Ca, Sr, Ba, Ra, Cu, Cd, Zn, Sc, Y, La, Ce, Pr, Nd, Sm, Eu, Tb, Ho, Er, Tm, Yb, Lu, and Pm. The synthesized magnetoradioactive metal nanoparticles can comprise magnetic metal hydroxide nanoparticles with a metal selected from the group consisting of: Gd, Mn, Fe, Dy, Co, Cr, Ni, Ru, Rh, Pd, Or, Ir, Pt, Pb, Sn, Ge, B, Al, Ga, In, Tl, Li, Na, K, Rb, Cs, Fr, Be, Mg, Ca, Sr, Ba, Ra, Cu, Cd, Zn, Sc, Y, La, Ce, Pr, Nd, Sm, Eu, Tb, Ho, Er, Tm, Yb, Lu, and Pm.

In one embodiment, the at least one radioactive atom comprises at least one of a positron emitter, a beta emitter, an alpha emitter, and a gamma emitter.

In one embodiment, the at least one radioactive atom is integrated to the synthesized magnetoradioactive metal nanoparticles.

In one embodiment, the magnetoradioactive metal nanoparticles further comprise a core including a crystal containing the at least one radioactive atom.

In one embodiment, a concentration of the at least one radioactive atom in the synthesized radioactive metal nanoparticles ranges between about 0.01 and about 5.0 mol-%.

In one embodiment, the at least one radioactive atom is in a concentration below about 1 wt % in the synthesized magnetoradioactive metal nanoparticles.

In one embodiment, the at least one radioactive atom is selected from the group consisting of: ¹¹C, ¹³N, ¹⁵O, ^{66/8}Ga, ⁶⁰Cu, ⁵²Fe, ^{61/2/4}Cu, ^{62/3}Zn, ^{70/1/4}As, ^{75/6}Br, ⁸²Rb, ⁸⁶Y, ⁸⁹Zr, ¹¹⁰In, ^{120/4}I, ¹²²Xe, ¹⁸F ^{99m}Tc, ^{123/5/131}I, ⁶⁷Ga, ¹¹¹In, ²⁰¹Tl, ³²P, ³⁹Sr, ¹⁶⁵Dy, ¹⁰⁹Pd, ¹⁶⁹Er, ¹⁰⁵Rh, ⁷⁷As, ¹⁴⁹Pm, ^{153/7}Sm, ¹⁷⁷Lu, ^{212/3}Bi, ^{186/8}Re, ⁶⁷Cu, ⁹⁰Y, ^{115m}In, ²¹¹At, ¹⁶⁶Ho, ¹⁹⁹Au, ¹¹¹Ag, ⁴⁷Sc, ^{193m}Pt, ^{117m}Sn, and mixtures thereof.

In one embodiment, the synthesized magnetoradioactive metal nanoparticles are covered by a stabilizer. The stabilizer can comprise a surfactant selected from the group consisting of: fructose, dextran, polyethylene glycol, dimercaptosucccinic acid (DMSA), citric acid, and mixtures thereof. The stabilizer can be biocompatible.

In one embodiment, the synthesized magnetoradioactive metal nanoparticles are smaller than about 100 nanometers. In another embodiment, the synthesized magnetoradioactive metal nanoparticles are between about 1 nm and about 50 nm. In still another embodiment, the synthesized magnetoradioactive metal nanoparticles are between about 1 nm and about 15 nm.

According to still a further general aspect, there is provided a multi-plasma apparatus for the synthesis of nanoparticles comprising: an electrolyte solution container; at least one cathode defining a plurality of spaced-apart gas channels having a gas output directed towards the electrolyte solution container; at least one anode electrically connectable to the at least one cathode; a gas supply connectable to the plurality of spaced-apart gas channels of the at least one cathode; and a power supply connectable to the at least one cathode and the at least one anode for creating a potential difference therebetween.

In one embodiment, the a plasma is generated in each one of the gas channels when gas flows therein and a potential difference between the at least one anode and the at least one cathode is created.

In one embodiment, the at least one cathode comprises a cathode body with a plurality of through holes in gas communication with the gas supply and defining at least a section of the gas channels. The at least one anode can comprise an anode body with a plurality of through holes in gas communication with the through holes defined in the cathode body. The multi-plasma apparatus can further comprise an insulating layer extending between the cathode body and the anode body and comprising a plurality of through holes in gas communication with the through holes defined in the anode body and the cathode body. The cathode body, the insulating layer, and the anode body can be superposed and the through holes defined in the cathode body, the anode body, and the insulating layer define the gas channels.

According to still a further general aspect, there is provided a multi-plasma apparatus, wherein the at least one cathode comprises a plurality of spaced-apart cathode capillary tubes spaced apart from one another and from the at least one anode and each one of the cathode capillary tubes has a tip pointing towards the electrolyte solution container, each one of the cathode capillary tubes has one of the gas channels defined therein. The cathode capillary tubes can comprise stainless steel and the at least one anode comprises graphite.

In one embodiment, the cathode capillary tubes are disposed around the at least one anode.

In one embodiment, the electrolyte solution container contains an aqueous electrolyte solution and the tips of the cathode capillary tubes are substantially equally spaced-apart from an interface of the aqueous electrolyte solution with air. The tips of the cathode capillary tubes can be spaced apart from the interface at least about 1 mm.

In one embodiment, the gas channels are substantially equally spaced-apart from the adjacent gas channels.

In one embodiment, the gas channels extend substantially parallel to one another.

In one embodiment, the electrolyte solution container contains an aqueous electrolyte solution.

In one embodiment, the at least one anode is at least partially immersable in an electrolyte solution contained in the electrolyte solution container.

In one embodiment, the at least one anode is mounted outwardly of an electrolyte solution contained in the electrolyte solution container.

In one embodiment, the multi-plasma apparatus further comprises at least one of a thermoelectric cooling device to allow for in situ temperature control and a pH probe linked to an automated pH adjustment system.

In one embodiment, the apparatus operates at atmospheric pressure.

In one embodiment, the gas supply comprises at least one of an inert gas supply and a reactive gas supply.

In one embodiment, the multi-plasma apparatus generates a plurality of micro-plasmas.

According to still a further general aspect, there is provided a method for synthesizing nanoparticles, the method comprising: providing at least one cathode defining a plurality of spaced-apart gas channels having a gas output directed towards an electrolyte solution contained in an electrolyte solution container; providing at least one anode electrically connected to the at least one cathode; supplying gas into the gas channels towards the electrolyte solution; and creating a potential difference between the at least one cathode and the at least one anode sufficient to ignite simultaneously plasmas in the gas channels.

In one embodiment, the at least one cathode comprises a cathode body with a plurality of through holes defining at least a first section of the gas channels and in which the gas is supplied. The at least one anode can comprise an anode body with a plurality of through holes in gas communication with the through holes defined in the cathode body and defining a second section of the gas channels. The method can further comprise providing an insulating layer extending between the cathode body and the anode body and comprising a plurality of through holes in gas communication with the through holes defined in the anode body and the cathode body and defining a third section of the gas channels and wherein the gas flows continuously in the first section, the second section, and the third section of the gas channels.

In one embodiment, the at least one cathode comprises a plurality of spaced-apart cathode capillary tubes spaced apart from one another and from the at least one anode and each one of the cathode capillary tubes has a tip pointing towards the electrolyte solution, each one of the cathode capillary tubes has one of the gas channels defined therein. The cathode capillary tubes can comprise stainless steel and the at least one anode comprises graphite.

In one embodiment, the electrolyte solution contained in the electrolyte solution container is an aqueous electrolyte solution.

In one embodiment, the gas channels are substantially equally spaced-apart from the adjacent gas channels.

In one embodiment, the gas channels extend substantially parallel to one another.

In one embodiment, the method can further comprise at least partially immersed the at least one anode in the electrolyte solution contained in the electrolyte solution container.

In one embodiment, the at least one anode is mounted outwardly of an electrolyte solution contained in the electrolyte solution container.

In one embodiment, the method further comprises cooling the electrolyte solution when plasmas are ignited.

In one embodiment, the method further comprises controlling a temperature of the electrolyte solution when plasmas are ignited.

In one embodiment, the method further comprises increasing an alkalinity of the electrolyte solution when plasmas are ignited.

In one embodiment, the method further comprises controlling a pH of the electrolyte solution when plasmas are ignited.

In one embodiment, the plasmas are ignited at atmospheric pressure and ambient air.

In one embodiment, the supplying step comprises supplying at least one of an inert gas and a reactive gas.

In one embodiment, a plurality of micro-plasmas are ignited simultaneously.

### BRIEF DESCRIPTION OF THE DRAWINGS

FIG. 1 is a schematic representation of a single-plasma apparatus in accordance with one embodiment;

FIG. 2 includes FIGS. 2a and 2b and are schematic representations of a multi-cathode plasma apparatus in accordance with one embodiment wherein FIG. 2a shows a top plan view of cathodes of the apparatus secured to a circular frame, FIG. 2b is a schematic representation of wiring and electrical components of the multi-cathode plasma apparatus wherein the cathodes are mounted in an array configuration;

FIG. 3 includes FIGS. 3a and 3b and are photographs of the single-plasma and multi-cathode plasma apparatuses shown in FIG. 1 and in FIG. 2 respectively during synthesis;

FIG. 4 is a photograph of the multi-cathode plasma apparatus shown in FIG. 2;

FIG. 5 is a schematic cross-sectional representation of another multi-plasma apparatus in accordance with one embodiment;

FIG. 6 is a graph showing a particle size distribution of magnetic MnOₓ nanoparticles synthesized with the single-plasma apparatus;

FIG. 7 includes FIGS. 7a and 7b and are TEM images (200 kV) of magnetic Mn-nanoparticles synthesized with MnCl₂ dissolved into a 2.5 g/L dextran aqueous stock solution with the single-plasma apparatus;

FIG. 8 includes graphs showing dynamic light scattering (DLS) hydrodynamic size measurement of magnetic Mn-containing nanoparticles (10 mMol MnCl₂*4H₂O, 2.5 mg/mL dextran, 30 minute plasma synthesis);

FIG. 9 includes FIGS. 9a and 9b and are DLS graphs of dialysed radioactive and magnetic metal nanoparticles (synthesis RM1) with intensity-weighted in FIG. 9a and with number-weighted profiles in FIG. 9b;

FIG. 10 includes FIGS. 10a and 10b and are DLS graphs of dialysed radioactive and magnetic metal nanoparticles (synthesis RM2) with intensity-weighted in FIG. 10a and with number-weighted profiles in FIG. 10b;

FIG. 11 includes FIGS. 11a, 11b, and 11c and are TEM images (120 and 200 kV) of magnetic Gd-nanoparticles synthesized with GdCl₃ in FIGS. 11a and 11b and Gd(NO₃)₃ in FIG. 11c dissolved into a 2.5 g/L dextran aqueous stock solution with the single-plasma apparatus;

FIG. 12 is a TEM image (120 kV) of magnetic Fe-nanoparticles synthesized with FeCl₃ dissolved into a 2.5 g/L dextran aqueous stock solution with the single-plasma apparatus;

FIG. 13 includes FIGS. 13a, 13b, 13c, 13d, 13e, and 13f and are relaxometric graphs indicating a MRI contrast agent potential of each dialyzed nanoparticle suspension synthesized from MnCl₂ in FIGS. 13a and 13b, from GdCl₃ in FIGS. 13c and 13d; and from Gd(NO₃)₃ in FIGS. 13e and 13f;

FIG. 14 are the MRI and PET images of duplicate samples (RM1 and RM2) of a magnetoradioactive contrast agent synthesized from MnCl₂ and ⁶⁴CuCl₂, using dextran as a surfactant; and

FIG. 15 include FIGS. 15a, 15b, 15c, 15d, 15e, and 15f and are TEM images wherein FIGS. 15a and 15b show magnetic MnOₓ nanoparticles, FIGS. 15c and 15d show magnetic GdOₓ nanoparticles, and FIGS. 15e and 15f show magnetic FeOₓ nanoparticles for FIGS. 15e and 15f and dissolved in a dimercaptosuccinic acid solution (10 mM) in FIGS. 15a, 15c and 15e and in a polyethylene glycol solution (PEG diacid (600 MW), 79.1 mM) in FIGS. 15b, 15d and 15f and produced with the multi-cathode plasma apparatus shown in FIG. 4.

It will be noted that throughout the appended drawings, like features are identified by like reference numerals.

### DETAILED DESCRIPTION OF THE PREFERRED EMBODIMENTS

A plasma-liquid electrochemistry process and associated apparatuses have been developed to allow the efficient production of ultra-fine, substantially uncontaminated nanoparticles made of metallic elements, within minutes. A suspension of ultra-small nanoparticles, which are at least one of magnetic and radioactive, is generated within minutes instead of 24 to 72 hours by traditional colloidal chemistry routes.

In the methodology developed, an atmospheric and adjustable plasma is generated at the interface of a solution containing metal ions, for instance metal salts. The plasma cathode is biased with respect to an anode, which is interacting with the cathode when a potential difference is created. Nanoparticles containing metallic elements, such as metal oxides, are generated at the plasma-liquid interface and they are readily covered with stabilizer molecules such as a surfactant or a surface ligand. For instance, a microplasma is projected towards an electrolyte solution containing metal ions and a metal oxide surfactant to produce magnetic and/or radioactive metal oxide nanoparticles. The microplasma(s) are ignited in confined spaced and, more particularly, in gas channels.

In one embodiment, the solution containing the metal ions is an aqueous electrolyte solution. However, one skilled in the art will appreciate that the solution can be a suitable non-inflammable solution.

In one embodiment, the plasma is generated at atmospheric pressure in an ambient air environment. However, one skilled in the art will appreciate that the plasma can be generated at atmospheric pressure in inert gas environment.

The nanoparticles are synthesized by generating a plasma at the interface of a liquid, including metallic ions and a stabilizer. Air (or a gas mixture) is present between the tip of the cathode and the interface of the liquid. The plasma is generated either by an inert gas (or gas mixture) flowing through the cathode, such as and without being limitative, argon, helium, or a reactive gas (or gas mixture), such as and without being limitative, H₂, N—₂, NH₃, and the like.

The stabilizer to prevent aggregation is either an electrostatic stabilizer having either positive or negative surface charges or a steric stabilizer covering the synthesized nanoparticles with polymers.

The stabilizer is a molecule, such as a surfactant or a surface ligand, added to the reactive bath to prevent particle coalescence and aggregation. For instance and without being limitative, the stabilizer can include carboxylic acids, acid halides, amines, acid anhydrides, activated esters, maleimides, isothicyanates, acetylacetonates, gold, silica precursors, a polyphosphate (e.g. calcium pholyphophates), an amino acid (e.g. cysteine), an organic polymer (e.g. polyethylene glycol/PEG, polyvinyl alcohol/PVA, polyamide, polyacrylat, polyurea), an organic functional polymer (e.g. 1,2-Distearoyl-sn-Glycero-3-Phosphoethanolamine-N-[Carboxy(polyethylene glycol)2000]ammonium salt), a biopolymer (e.g. polysaccharide like dextran, xylan, glycogen, pectin, cellulose or polypeptide like collagen, globulin), cysteine or a peptide with high cysteine content or a phospholipid. In one particular and non-limitative embodiment, the surfactant is fructose, dextran, polyethylene glycol, dimercaptosucccinic acid (DMSA) or citric acid. The stabilizer prevents nanoparticle agglomeration in the liquid and slows the nanoparticle growth.

The plasma acts as an electron supply at the liquid interface. The mechanism of nanoparticle nucleation and growth involves (a) an interaction between electrons and chemical species in the liquid and (b) an intermediate step taking place in the liquid, by which the metal ions bind to oxygen species, thereby forming nanoparticles with a high content in metallic elements (for instance, metal oxide, metal hydroxide, and metal phosphate nanoparticles having a significant metallic element content). The plasma is generated at atmospheric pressure. More particularly, the synthesized nanoparticles can be magnetic, radioactive, or magnetoradioactive, i.e. magnetic and radioactive.

The distance between the electrodes, i.e. the anode(s) and the cathode(s), is a relatively important design variable for the plasma apparatus. More particularly, the location of the smallest distance “y” between the closest portions of the electrode(s) used in the plasma apparatus will determine the breakdown voltage necessary to ignite the plasma. Between a metal cathode and the liquid interface, “y” should be greater than a distance “x” between the tip(s) of the cathode and the air/solution interface in order to prevent an undesirable arc or formation of an unwanted corona or plasma occurring between the electrodes. The use of a dielectric barrier or the use of a jet configuration can also be used to alleviate the risk of undesirable arc or formation of an unwanted corona. One skilled in the art will appreciate that various electrode design(s), electrode location(s) and electrode interaction(s) are possible.

To synthesize radioactive nanoparticles, radioactive ions are added to the electrolyte solution including the metallic ions and the surfactant. For instance and without being limitative, a radioactive salt can replace a fraction of the metallic salt.

The apparatus includes a cathode which lies above the liquid interface and an electrode acting as an anode, such as a graphite rod, entirely or partially immersed in an aqueous solution containing metal ions and a surfactant molecule. In an alternative embodiment, both electrodes, i.e. the anode and the cathode(s), are mounted outside the electrolyte solution to provide a jet configuration. One or several capillary tubes, which can act as cathode(s) and can be made of stainless steel or act as dielectric barrier(s) and can be made of plastics or glass, is/are placed a few millimeters from the surface of the electrolyte solution, pointing downwards. Gas flows in the capillary tubes, then the cathode(s) is/are biased with respect to the anode, resulting in the creation of a plasma between (a) the cathode(s) and the surface of the electrolyte solution in the case of the anode immersed in the liquid or (b) between the cathode and the anode in the case of a jet configuration. Interaction between plasma electrons and chemical species in the liquid causes nanoparticles to nucleate and grow in the fluid. Since the cathode(s) is/are not immersed in the solution, the nanoparticles do not adhere onto the surface. Therefore, they remain in suspension in the liquid provided that the liquid contains a stabilizer, such as a surfactant and a surface ligand. The stabilizer role is to cover the surface of the nanoparticles in situ. During the synthesis process, agitation is provided by the flux of gas flowing through the cathode, which helps homogenize the nanoparticle nucleation. Other means of agitation can be used, provided that the distance between the cathode and the surface of the liquid is not subject to severe changes that could lead to the disruption of the plasma.

The apparatus includes at least one power source connected electrically between to the cathode(s) and the anode. In one embodiment, the power source is a source of volts and amps such as a transformer. The power source can be a direct current (DC) power source or an alternative current (AC) power source. In one non-limitative embodiment, the power source is an AC power source with a low frequency.

One skilled in the art will appreciate that the electrodes, i.e. the anode(s) and the cathode(s), can be of any suitable composition and suitable physical configuration (e.g., size and shape) which results in the creation of a desirable plasma.

In one embodiment, the synthesized nanoparticles are smaller than about 100 nanometers (nm) and, in an alternative embodiment, the synthesized nanoparticles are between about 1 to 100 nm. In another alternative embodiment, the synthesized nanoparticles are between about 1 to 10 nm. In another alternative embodiment, the synthesized nanoparticles are less than about 5 nm.

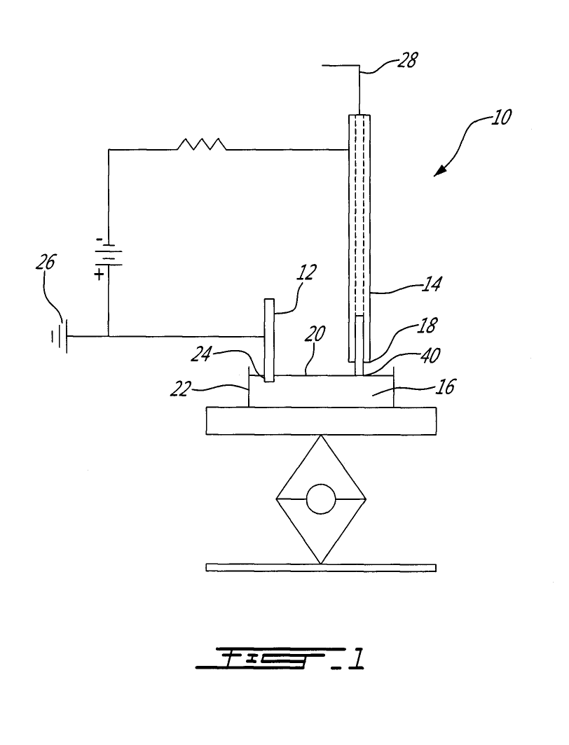

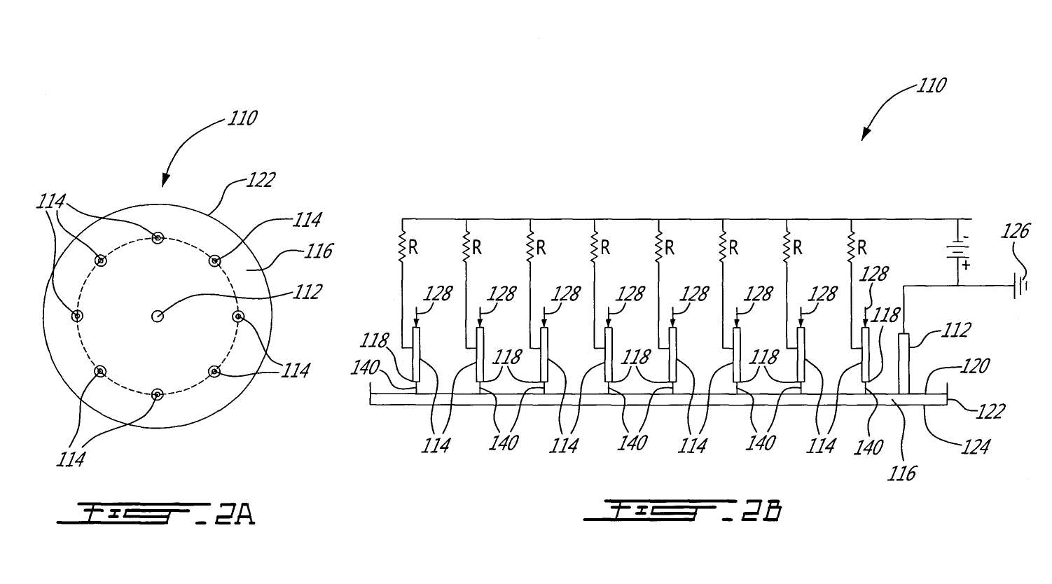

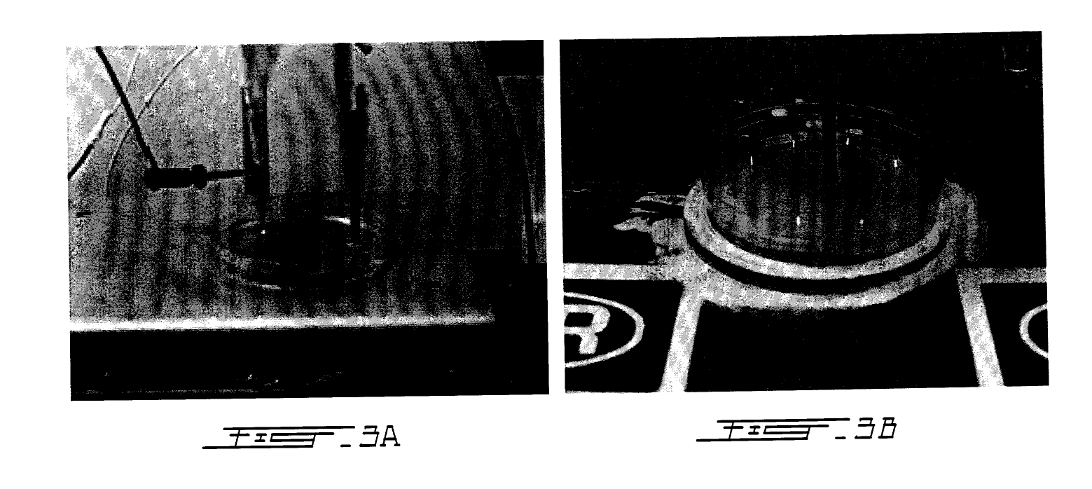

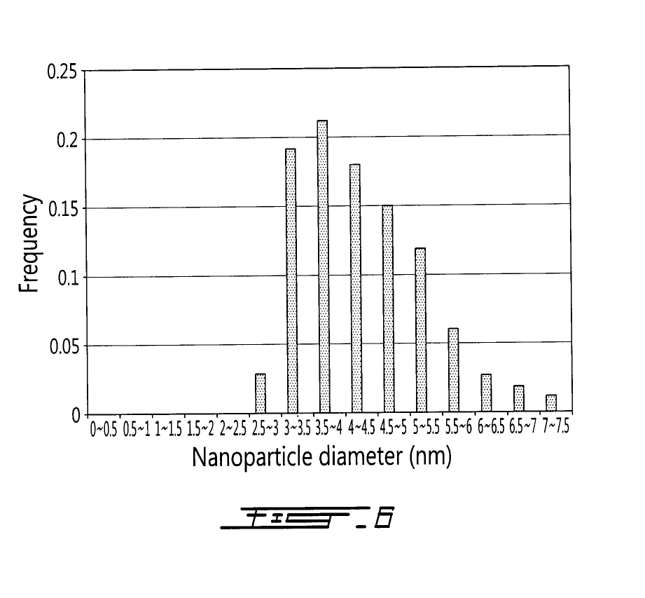

To synthesize magnetic and magnetoradioactive nanoparticles, three apparatuses were developed. A first apparatus is shown in FIGS. 1 and 3a and includes a single cathode/plasma, which is similar to a previously reported plasma-liquid electrochemical cell for the synthesis of Ag and Au particles (C. Richmonds, et al., Applied Physics Letters 2008, 93). A second apparatus, shown in FIGS. 2 and 3b, is a multi-plasma apparatus and a third apparatus, shown in FIG. 5, is another multi-plasma apparatus. The second and the third apparatuses were built to allow the fast synthesis of a concentrated nanoparticle suspension. They can include a thermoelectric cooling device to allow for in situ temperature control and a pH probe linked to an automated pH adjustment system.

As used herein, the terms “single-plasma electrochemical cell or apparatus” is intended to include any electrochemical cell or apparatus wherein a single plasma is generated and directed towards the electrolyte solution interface. In one embodiment, the single-plasma electrochemical cell or apparatus includes only one cathode and the single plasma is generated between the cathode and the surface of the electrolyte solution.

As used herein, the terms “multi-plasma electrochemical cell or apparatus” is intended to include any electrochemical cell or apparatus wherein more than one plasma is generated simultaneously and directed towards the electrolyte solution interface. For instance, an array of microplasmas is generated in a multi-plasma electrochemical cell. In one embodiment, the multi-plasma electrochemical cell or apparatus includes more than one cathode and one plasma is generated between each one of the cathodes and the surface of the electrolyte solution. In an alternative embodiment, the multi-plasma electrochemical cell or apparatus includes one or more cathodes and more than one plasma is generated by each cathode and directed towards the surface of the electrolyte solution. For instance, the cathode is designed to produce more than one simultaneously gas flow towards the electrolyte solution and which generates multiple plasmas.

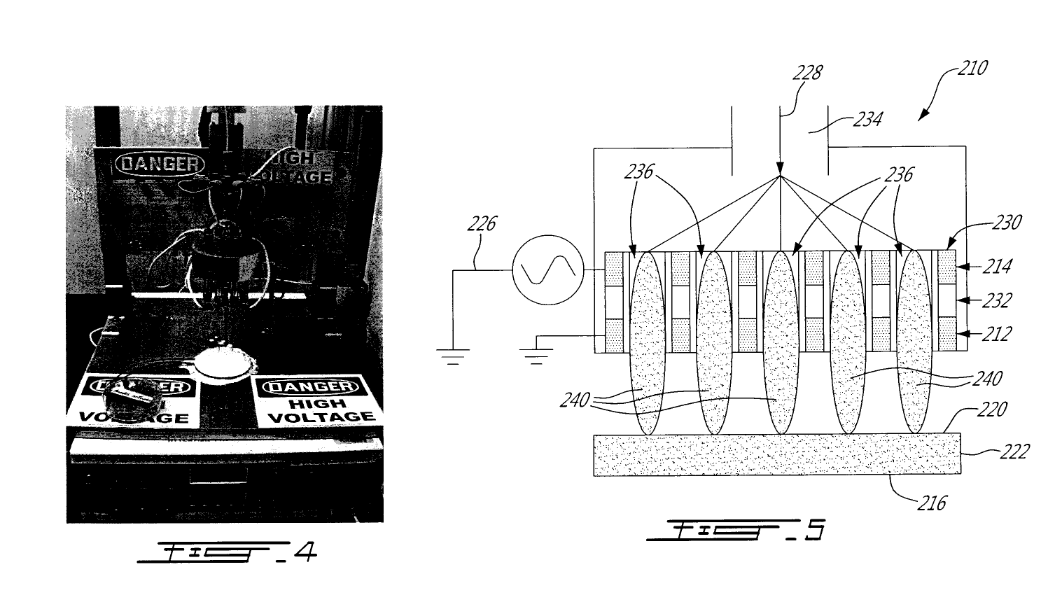

The two apparatuses shown in FIGS. 2, 3b, 4, and 5 are both variants of a multi-plasma apparatus. To distinguish both apparatuses, the apparatus shown in FIGS. 2, 3b, and 4 will be referred to as the multi-cathode apparatus since it includes a plurality of cathodes wherein each one of the cathodes generates one plasma. The apparatus shown in FIG. 5 will be referred to as the multi-plasma apparatus and includes only one cathode having a grid like shape with through holes.

Experiments were carried out with the first and the second apparatuses respectively shown in FIGS. 1 to 4. For the tests carried out, the plasma-liquid electrochemical cell 10 included a graphite electrode (3.05 mm diameter, Sigma-Aldrich Canada) acting as an anode 12, and at least one stainless-steel capillary, acting as a cathode 14 (Gauge 26 needle, 260 μm ID, 460 μm OD, point style 3 (PT3), Hamilton Company USA or grade 25 needles, 241 μm ID, Terumo Medical Products, Japan; or grade 23 needles, 318 μm ID, BD, Franklin Lakes, N.J.), placed a few millimeters away, both pointing downwards to an electrolyte solution 16 containing metal ions and a surfactant molecule. The tip 18 of the stainless steel capillary did not touch the surface 20 of the liquid 16, contained in a Petri dish 22. A gas, such as argon, flowed in a gas channel defined in the biased cathode, and this generated a plasma 40 between the tip of the cathode 14 and the surface 20 of the liquid (or the electrolyte solution). Because of the Pendel effect (A. Gunther-Schulze, Zeitschrift Fur Physik 1923, 19, 313-332), which refers to an oscillatory movement of electrons between the walls of the cathode, the geometrical configuration of the hollow cathode produced plasma 40 of high electron density. The diameter of the hollow cathode was fixed by considering the critical value pD (p: gas pressure in the capillary; D: inner diameter) (H. Helm, Zeitschrift Fur Naturforschung Section a-a Journal of Physical Sciences 1972, A 27, 1812-1820). During the plasma-liquid electrochemical process, metal ions in the liquid were reduced by plasma electrons, causing nanoparticles to nucleate and grow in the fluid. The surface of nanoparticles was systematically stabilized by the presence of surfactants. In the examples described below, dextran, dimercaptosuccinic acid and polyethylene glycol were used as surfactants. However, one skilled in the art will appreciate that other surfactants can be used as mentioned above.

In the embodiment described and shown in FIGS. 1 to 4, the anode 12 is mounted outwardly of the Petri dish 22 and only its tip 24 is immersed in the solution. As previously mentioned, in an alternative embodiment, the anode can be entirely immersed. For instance and without being limitative, the anode can be positioned in the bottom of the Petri dish, or any other appropriate container. In another alternative embodiment, shown in FIG. 5, the anode can be mounted completely outside the liquid phase (or the electrolyte solution). In the embodiment shown in FIG. 5, the anode is mounted above the electrolyte solution, spaced-apart from the cathode but in electric interaction therewith. In an alternative embodiment (not shown), the anode can be mounted anywhere close to the Petri dish, or any other appropriate reservoir, provided that it is in electrical interaction with the cathode and a plasma is generated and projected towards the liquid interface. In one non-limitative embodiment, the shortest distance between the anode and the cathode is smaller than about 10 centimeters. For instance and without being limitative, in a jet configuration, the anode can be positioned close to the cathode to generate the plasma in the gas flow, with the plasma then projected on the liquid interface.

Single-Plasma Operation:

A schematic representation of a single cathode device generating a single plasma is given in FIG. 1. A DC power source 26 that can provide up to 3000 V and 150 mA (Pharmacia, ECPS 3000/150) was used. A 70 kΩ resistance (R) was fixed in serial in the circuit. The cathode 14 was connected to an argon supply 28, and the metal ion solution was placed in a 6-cm diameter Petri dish 22. The distance between the liquid and the tip of the hollow cathode was adjusted to allow the creation of a plasma. All single-plasma nanoparticle synthesis procedures were performed as follows: first, the anode 12 was partially immersed (^{˜}2 mm) in the electrolyte 16; then, the tip 18 of the hollow cathode 14 was brought to about 1-2 mm from the liquid. Argon was let flowing into the gas channel defined in the cathode at 25 SCCM. The required bias voltage needed to sustain the plasma ranged between 1100 and 1250 V (at 10 mA). Right after plasma ignition, the current intensity was lowered to 6-7 mA. In single-plasma experiments, all solutions were plasma-treated for 30 minutes. A picture of the plasma electrochemical cell 10 in operation is given in FIG. 3a.

One skilled in the art will appreciate that the power source can either be DC or AC (i.e. high-voltage nanopulses, low frequency (LF), radio-frequency (RF) and microwaves (MW)). Several power sources can also be combined.

Multi-Plasma System Operation:

In the purpose of enhancing the efficiency and productivity of the plasma-liquid electrochemical cell, the single-plasma apparatus was upgraded to a multi-plasma prototype. In the alternative embodiment of the apparatus shown in FIGS. 2, 3b, and 4, the features are numbered with reference numerals in the 100 series which correspond to the reference numerals of the previous embodiment.

In one particular configuration, eight (8) cathodes 114, each having a gas channel defined therein, were positioned in a circle shaped around a graphite anode 112 as shown in FIGS. 2a, 3b, and 4 to obtain a multi-cathode electrochemical cell 110. In an alternative configuration, the cathodes could be positioned in an array as shown in FIG. 2b. Alternatively, these configurations can also be easily adapted to provide 4 or 6 cathodes. A constant distance was kept between the tip of the cathodes and the liquid interface. In order to prevent excessive heating of the solution during the plasma treatment, the Petri dish 122 was seated on a thermoelectric module (Vₘₐₓ=31.7V, Iₘₐₓ=6.1A, Qₘₐₓ=119.0 W, ΔTₘₐₓ=70° C.; TE technology inc, Traverse City, Mich.) and thermal transfer was ensured by a heat sink compound (Thermalcote, part. #: 250, Thermalloy, Dallas, Tex.). The synthesis process consumes hydroxide groups, resulting in the rapid acidification of the solution. Under acidic conditions, metal oxide nanoparticles are more susceptible to dissolution. Therefore, lowering the temperature of the solution is necessary to prevent the degradation of the resulting metal oxide cores. A picture of the plasma electrochemical cell 110 in operation is given in FIG. 3b wherein plasmas 140 are created between the cathodes 114 and the electrolyte solution interface 120.

In one first non-limitative embodiment, the temperature of the solution should be maintained between about 0° C. and about 50° C. and, in another non-limitative embodiment, between about 25° C. and about 40° C.

The pH of the solution should be maintained in a range determined in accordance with the Pourbaix diagram of the synthesis carried out. In one non-limitative embodiment, the pH of the solution should be maintained between about 5 and about 7.

As mentioned above, the consumption of —OH groups in the electrolyte solution and the resulting acidification of the solution may create synthesized nanoparticle dissolution or aggregation problems. pH adjustments can be carried out by either adding NaOH solutions in a dropwise manner in the nanoparticle suspensions during the synthesis or by using —OH containing stabilizer molecules such as and without being limitative carboxymethyl-dextran or other saccharides.

All multi-plasma nanoparticle synthesis procedures were performed as follows: the cathodes 114 were brought to about 1-2 mm from the liquid; argon was let flowing into the cathode at 25 SCCM per cathode, for a total flow of 200 SCCM while the DC power source 126 kept the cathodes biased to 1250 V (55 mA). Right after plasma ignition, the current intensity was lowered to 40 mA. In multi-plasma experiments, all solutions were plasma-treated during 30 seconds to 5 minutes.

It is appreciated that the apparatus 110 can include more or less cathodes 114 and that several cathode configurations are possible providing that necessary adjustments to the system are made.

In one non-limitative embodiment, the gas channels defined in the cathodes 14, 114 have a diameter ranging between 50 μm and 5 mm.

Thus, with the apparatus shown in FIGS. 2, 3b, and 4, multiple plasmas were generated through multiple cathodes.

Multi-Plasma System:

FIG. 5 shows an alternative apparatus 210 to generate multiple plasmas 240. In this alternative embodiment, the features are numbered with reference numerals in the 200 series which correspond to the reference numerals of the previous embodiments. The apparatus has a micro-hollow dielectric barrier discharge (MH-DBD) jet configuration, with microplasmas generated in multiple through holes and projected on the liquid interface. The apparatus includes a plasma generating device 230 with two stacked micromachined metal electrodes 212, 214, used respectively as an anode body 212 and a cathode body 214, with an insulating (dielectric) film (or layer) 232 extending in-between. A plurality of through holes 236 is defined in the superposed electrodes 212, 214 and insulating layer 232. The through holes defined in each one of the superposed layers are in register with another one of the through holes defined in another one of the superposed layers. Thus, the injected gas first flow in a first common channel 234 and is then divided in a plurality of gas flows, each flowing in a respective one of the through holes 236, or gas channels which are spaced-apart from one another, defined in the superposed electrodes 212, 214 and insulating layer 232. The plasma generating device 230 thus has a grid shape design with multiple through holes. Each one of the gas channels has a gas output directed towards the electrolyte solution. Thus, gas is injected towards the electrolyte solution in a showerhead design and generates a series of in-plane microdischarges. The discharge occurs inside each coaxial hole (or through hole) 236 and generates plasma jets onto the liquid interface 220. By using such a configuration, the liquid is no longer in direct contact with the anode 212. In other words, the anode 212 is spaced-apart from the electrolyte solution 216, which alleviates risks of contamination from the anode material in the synthesized nanoparticles. Moreover, the decoupling of the plasma generation and the nanoparticle production at the plasma-liquid interface 220 offered by this embodiment result in the liquid properties having no effect on the plasma properties and stability conditions. Due to the insulating layer 232 extending between the anode 212 and the cathode 214, DC current cannot be used. Instead, a low frequency AC current or other suitable energy supplies can be used.

In one non-limitative embodiment, the gas channels 236 defined in the plasma generating device 230 have a diameter ranging between about 50 μm and about 5 mm. In one non-limitative embodiment, the distance between two adjacent gas channels 236 range between about 50 μm and about 15 mm. In another non-limitative embodiment, the distance between the lower surface of the anode 212 and the interface of the plasma-liquid interface 220 ranges between about 0.5 cm and about 10 cm.

For instance and without being limitative, copper, aluminum, stainless steel or transparent conducting oxides (TCO such as ITO ((Indium tin oxide) can be used as electrode materials, Dielectric materials used as insulating materials can include, without being limitative, alumina, glass, PTFE, PVDF or any other ceramics or polymers with good dielectric properties.

Lead-Shielded Bench for the Synthesis of Radioactive Nanoparticles:

Metal nanoparticles containing the positron emitter ⁶⁴Cu were synthesized using the multi-cathode (or multi-plasma) device shown in FIGS. 2, 3b, and 4. For this procedure, the plasma-liquid electrochemical cell was placed in a ventilated hood shielded with 5 cm-thick lead walls, and equipped with a 10 cm-thick leaded glass allowing the manipulator to visualise plasma-liquid electrochemical synthesis experiments. Tungsten syringe shields (Biodex, Shirley N.Y.) were used to protect the manipulators during the synthesis and dialysis procedures. A dose calibrator was inserted through the bench surface, and allowed for direct and convenient radioactivity monitoring of samples, syringes, and contaminated tools, vials, and dialysis membranes.

Synthesis of Non-Radioactive Magnetic Metallic Nanoparticles with the Single-Plasma Electrochemical Cell with Dextran as Stabilizer

Magnetic metallic elements are elements expressing a significant permanent magnetic moment. Magnetic metallic elements include mainly the transition iron group (3d) as well as rare-earth elements (f4). Among magnetic elements figure iron (Fe), cobalt (Co), nickel (Ni), chromium (Cr), manganese (Mn), and rare earth metals gadolinium (Gd) and dysprosium (Dy). In this specification, the term “magnetic metallic element” is intended to include ferromagnetic metals (or ferromagnetic elements) such as Fe, Co, Cu, Cr, and Ni but also metallic elements expressing a magnetic moment typically above 0.5 [symbol] _{B}.

Thus, to synthesize magnetic metal nanoparticles, the electrolyte solutions include metal ions of at least one magnetic (including ferromagnetic and paramagnetic) metals including but not limited to Fe, Co, Ni, Mn, Cr, Gd, Cu, Eu, and Dy.

Prior to all experiments, electrolyte solutions were prepared, containing metal ions and dextran. First, a solution of 2.5 mg/mL of dextran (5000 MW, Carbomer, San Diego Calif.) was prepared using nanopure water (18.2 Macm Barnstead Nanopure water system). This stock solution was sonicated 30 minutes and then centrifuge-filtered 30 minutes (50 mL, 0.2 μm, Sartorius Stedim biotech, 400G). Aqueous metal ion precursor solutions were prepared in parallel by dissolving in nanopure water MnCl₂*4H₂O, Gd(NO₃)₃*6H₂O, GdCl₃*6H₂O or FeCl₃*6H₂O (all of purity >99.9%, and from Sigma-Aldrich, Canada). Then, the precursor solutions were mixed with the dextran stock solution (1:4 vol./vol.), resulting in electrolyte solutions containing 1 mg/mL of dextran, and 0.1, 1, 5, 10, 25 or 100 mM of metal ions. After a 30-minute sonication, the resulting electrolyte solutions were transferred into Petri dishes for plasma synthesis.

One skilled in the art will appreciate that other metal ion precursors such as and without being limitative metal chlorides, metal nitrates, metal acetates, and metal organometallics, and mixtures thereof, can be used.

Centrifugation, Filtering, Dialysis:

As-synthesized plasma-treated products were centrifuge-filtered (0.2 μm, 400 G, 15 minutes). Then, four (4) mL of as-synthesized suspensions were placed in 10 000 MW dialysis membranes (Spectra/Por #6, Rancho Dominguez, Calif.), and purified in nanopure water (18.2MΩ·cm, 1 L) for 21 hours to eliminate free metal ions and excess dextran. The water was changed 3 times and the sample to volume ratio was kept to at least 1:1000. After dialysis, samples were transferred to 15 mL tubes and centrifuged at 1100 G for 15 minutes to eliminate coarse agglomerates.

Particle Size Study:

The hydrodynamic diameter of aqueous suspensions of as-dialyzed nanoparticles was measured by dynamic light scattering (DLS, Malvern Zetasizer 173°), and compared with the results from transmission electron microscopy (TEM) analysis. For TEM studies (120 keV: Jeol JEM-1230; 200 keV: JEM-2100F) 3 μL drops of as-synthesized and dialyzed suspensions of nanoparticles were dried on carbon-coated copper grids (Canemco-Marivac).

¹H NMR Relaxivity:

To confirm the potential of plasma synthesized Mn and Gd nanoparticles as efficient MRI contrast agents, fractions of as-dialyzed Mn and Gd-containing suspensions were distributed in 6.0 mm NMR tubes and diluted with nanopure water. Then, the longitudinal and transversal relaxation times (T₁ and T₂) of ¹H protons were measured with a dedicated TD-NMR relaxometer (Bruker Minispec 60 mq, 60 MHz, 20° C.).

Metal Concentration Measurement in Dialyzed Suspensions:

Samples of the nanoparticle suspension were digested in nitric acid and hydrogen peroxide 30% (both trace metal grade, from Sigma-Aldrich). Then, the concentration of metals in dialyzed suspensions was precisely measured by graphite furnace atomic absorption spectrometry for Mn (GF-AAS, Perkin Elmer AAnalyst 800) or with inductively coupled plasma mass spectrometry for Gd (ICP-MS, Perkin Elmer Elan 6000). Finally, relaxation rates (1/T₁ and 1/T₂) were plotted against metal concentration values, and relaxivities (r₁ and r₂) were calculated as follows:

$$\frac{1}{{T}_{i}} = \frac{1}{{T}_{id}} + {r}_{1 , 2} [ Gd , Mn , Fe ] i = 1 , 2$$

in which T_{id} refers to the relaxation times of the matrix, [Gd, Mn, Fe] to the molar concentration of Gd, Mn or Fe, and r₁ and r₂ to the longitudinal and transversal relaxivities, respectively. The efficiency of a paramagnetic, ferromagnetic or superparamagnetic substance to increase the relaxation of ¹H protons in water is referred to as the “relaxivity” (A. E. Merbach, et al., The chemistry of contrast agents in medical magnetic resonance imaging, Wiley, Chichester; N.Y. 2001.):

$$\frac{1}{{T}_{i}} = \frac{1}{{T}_{id}} + {r}_{1 , 2} [ Gd , Mn , Fe ] i = 1 , 2$$

T_{od} refers to the relaxation times of the matrix, [Gd, Mn, Fe] the molar concentration of Gd, Mn or Fe, r₁, and r₂ the longitudinal and transversal relaxivities, respectively.

Synthesis of Non-Radioactive Magnetic Metallic Nanoparticles with the Multi-Cathode Electrochemical Cell and Dimercaprosuccinic Acid and Polyethylene Glycol as Stabilizers

Experiments to demonstrate the synthesis of magnetic metallic nanoparticles with other surfactants (or stabilizers) were also carried out. More particularly, MnOₓ, GdOₓ, and FeOₓ as magnetic metallic oxides nanoparticles were synthesized with dimercaprosuccinic acid (DMSA) and polyethylene glycol (PEG) as surfactants. The experimental procedure was the same as the one described above for the synthesis of non-radioactive magnetic metal nanoparticles with the single-plasma electrochemical cell (or single-cathode cell) using dextran as surfactant. However, DMSA (meso-2,3-dimercaptosuccinic acid, ^{˜}98%, Sigma-Aldrich, Canada) and/or PEG (Poly(ethyleneglycol)bis(carboxymethyl)ether, Mn ^{˜}600, Sigma-Aldrich, Canada) were used as surfactants instead of dextran. When using DMSA or PEG as surfactants, the electrolyte solutions contained from 8.2 to 10 mM of DMSA or from 15.9 to 79.4 mM of PEG, and a salt concentration ranging from 0.82 to 10 mM.

The centrifugation, filtering, and dialysis procedures are the same as the one carried out for the synthesis of non-radioactive magnetic metal nanoparticles with the single-plasma electrochemical cell using dextran as surfactant and detailed above.

The nanoparticles synthesized were studied with a transmission electron microscopy (TEM) analysis (120 keV: Jeol JEM-1230). More particularly, 3 μL drops of as-synthesized suspensions of nanoparticles were dried on carbon-coated copper grids (Canemco-Marivac).

Synthesis of Radioactive and Magnetic Metal Nanoparticles

Radioisotope Solution:

The radioisotope ⁶⁴Cu(I_{β+}=17%, I_{β-}=39%, I_{EC}=43%; half-life: 12.7 h) is an important emerging biomedical radionuclide. Its half-life is well suited for molecular and cellular imaging applications requiring the monitoring of biological processes over periods of time longer than that covered by most PET nuclides. Its positron energy spectrum is comparable to that of ¹⁸F (¹⁸F half-life: 110 minutes), resulting in high PET spatial resolutions. In this study, ⁶⁴Cu was prepared at Université de Sherbrooke with a TR-19 cyclotron (Advanced Cyclotron Systems, Richmond, BC, Canada) by the ⁶⁴Ni(p,n)⁶⁴Cu reaction, using an enriched ⁶⁴Ni target electroplated on a solid rhodium disc (22 mm diameter, 1 mm thickness) (S. K. Zeisler, et al., Journal of Radioanalytical and Nuclear Chemistry 2003, 257, 175-177). ⁶⁴Ni was purchased from Isoflex (San Francisco, Calif., USA). ⁶⁴CuCl₂ was recovered from the target material following an established protocol (D. W. McCarthy, et al., Nuclear Medicine and Biology 1997, 24, 35-43), and the aqueous recovery solution was evaporated to dryness. The radioisotope was then dissolved into 0.5 mL of nanopure water, and immediately shipped to the laboratory in 2.5 h (decay factor: 0.8725). The total activity of the vial was 4.65 mCi (in 0.5 mL), at the beginning of the experiments (time=0 min), and the pH of the solution was ^{˜}1 upon delivery.

In addition to ⁶⁴Cu (⁶⁴Cu half-life: 12 h), it is appreciated that other radionuclides (or radioistopes), either positron, beta, alpha or gamma emitters, can be used such as and without being limitative, ⁸⁶Y (14 h), ¹²⁴I (4.2 days), ⁸⁹Zr (3.2 days), ¹⁸F (110 minutes), ⁶⁷Ga, ¹²³I, ¹¹¹In, etc.

Precursor Solution and Synthesis:

Prior to radioactive synthesis, the multi-cathode equipment was placed in the shielded vented bench. The precursor solution containing Mn²⁺ ions and dextran was prepared as described above, and 10 mL of that solution were placed in a Petri dish. Just before the synthesis, 100 μL of the ⁶⁴CuCl₂ solution were added to the dish, and the pH was equilibrated to 6.5 by adding 140 μL of 0.3 M NaOH. The synthesis was repeated (duplicate) and the two samples were named RM1 and RM2 (synthesized at time=50 min and time=90 min, respectively).

Standard ⁶⁴CuCl₂ and ¹⁸F-FDG Samples for Activity Assessment:

At time=0, samples of the ⁶⁴CuCl₂ solution were pipetted into 2 mL centrifuge tubes, and used as standards. Samples containing 2, 5, 10 (triplicate) and 20 μL of the radioactive solution were filled up to 2 mL with nanopure water and measured in the dose counter. The dose calibrator indicated activities of 18.8, 45.7, 90.4, 91.4, 90.5, and 181 μCi for each sample, respectively, corresponding to 101, 98.3, 97.2, 92.3, 97.3 and 97.3% of radionuclide recovery (average recovery factor: 97.2%). At the time of PET imaging (time=6 h), the activity in the vials had decayed to 13.5, 32.9, 65.2, 65.9, 65.2 and 130 μCi, respectively. As expected, a small fraction of ⁶⁴Cu was lost in the vials and the syringe.

Dialysis and Dilutions:

As-synthesized samples RM1 and RM2 were pipetted in 10 000 MW floating dialysis membranes (10 000 MW dialysis membranes (Spectra/Por #6, Rancho Dominguez, Calif.)), and dialyzed for 2 h30 h in 1.5 L of nanopure water, under constant agitation. The water was refreshed after 1 h30. Solutions RM 1 and RM2 were extracted from the membranes, and placed in 0.5 mL centrifugation microtubes (Eppendorf). For the relaxometric study, they were appropriately diluted with nanopure water to reach 10, 25, 50 and 100% v./v. (as described above). Fractions of these were analyzed in GF-AAS to measure the exact Mn concentration in the dialyzed products.

MRI:

The dilutions of magnetoradioactive suspensions were placed in microtube racks filled with nanopure water (time=23° C.). They were inserted in a 5.5 cm diameter small-animal RF transmit and receive coil. The samples were imaged with a M2 ASPECT 1T MRI system (ASPECT magnet technologies Inc, Netanya, Israel) using a bidimensional (2-D) spin echo sequence (3 NEX; 2 mm slice thickness, 0.2 mm interspace), with a FOV of 80 mm and a 256×256 matrix. The samples were successively scanned in both T₁ (TE: 6.8 ms; TR: 700 ms) and T₂-weighted imaging (TE: 25 ms; TR: 2217 ms).

PET and Dose Counter:

Prior to MRI visualization, the series of magnetoradioactive suspensions were imaged at time=6 h, with a PET/CT Siemens Biograph 6 system. The series of vials were inserted in a bowl filled with nanopure water (as described for MRI studies), which allowed for a more complete positron annihilation. Diluted ⁶⁴CuCl₂ solutions were also inserted in the positioning rack to allow direct comparisons of the activity. The samples were imaged using a brain protocol; a preliminary CT scan (15 seconds) was used for attenuation correction prior to PET scanning (10 minutes). The voxel size was 2.34×2.34×3.0 mm. All radioactive samples were measured at the dose counter after PET imaging procedures.

Results

Table 1 summarizes the parameters that were used in each procedure: precursor salt and concentration, plasma cathode configuration, treatment time and the final concentration after dialysis. Then, the longitudinal and transversal relaxivities are resumed, as well as r₂/r₁ relaxommetric ratios. The TEM studies have clearly and unambiguously revealed the presence of a large amount of ultrafine nanoparticles that are similar to MnO and Gd₂O₃ nanoparticles synthesized with the conventional methods (thermal decomposition and polyol colloidal synthesis) (R. Bazzi, et al., Journal of Luminescence 2003, 102, 445-450; J. Park, et al., Nature Materials 2004, 3, 891-895). Because the synthesis reaction involved the consumption of hydroxyl groups, which was confirmed by a sharp acidification of the solution during the plasma treatment, the nanoparticles were expected to reveal a metallic oxide composition. Therefore, the synthesized products are referred as MnOₓ, GdOₓ and FeOₓ nanoparticles below.

More particularly, Table 1 shows the longitudinal (r₁) and transversal relaxivities (r₂) of plasma-synthesized nanoparticle suspensions produced with the single-plasma apparatus and with dextran as stabilizer. The nanoparticles were produced by using metal salts diluted in 2.5 g/l dextran solutions. The relaxivities of metal ions diluted in the dextran stock solution are also shown.

Size-Distribution Study of MnOₓ Magnetic Metal Nanoparticles:

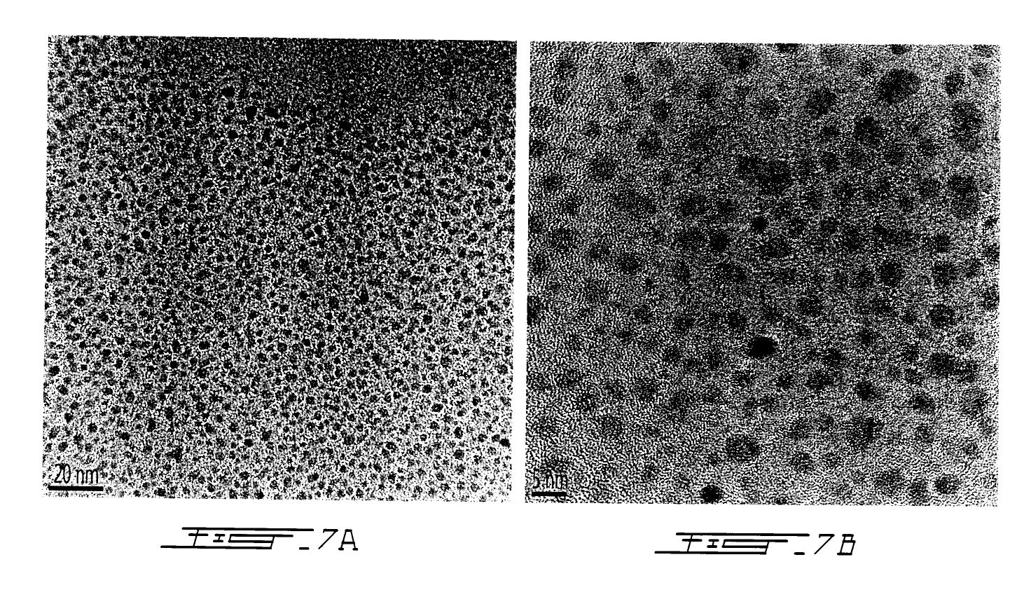

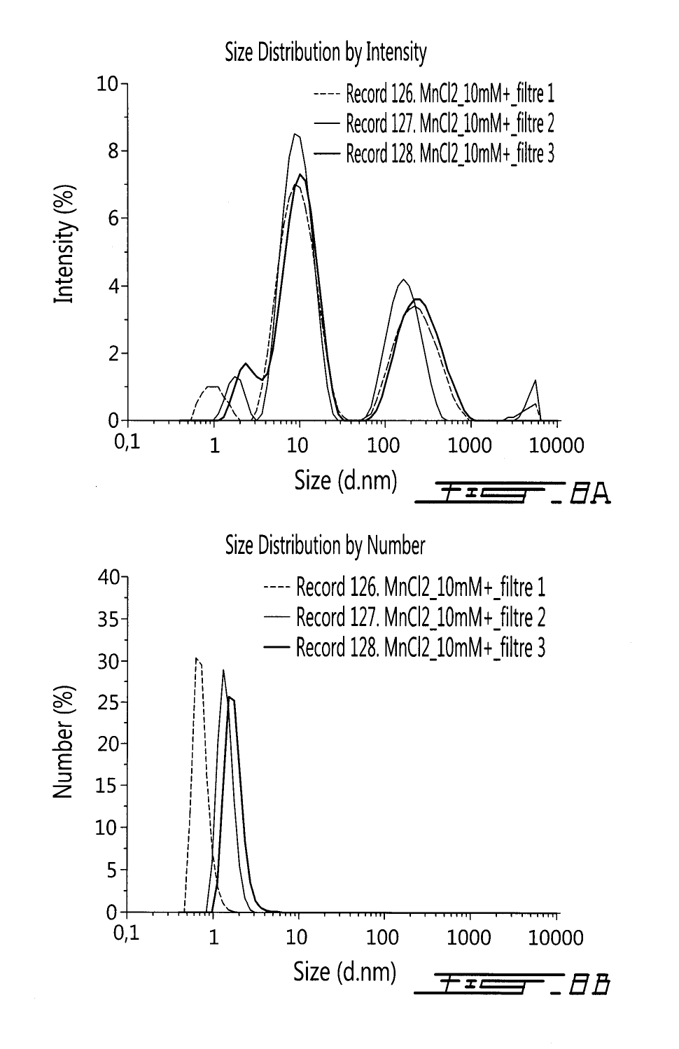

In the examples, manganese chloride was used as a precursor for the synthesis of all MnOₓ nanoparticles. The single-plasma device was used with three different precursor concentrations (0.1, 5 and 100 mM Mn) whereas in multi-plasma experiments, the concentration of Mn salt was fixed to 10 mM. As shown in FIG. 7, the MnOₓ nanoparticles synthesized from 5 mM solutions were homogeneously spherical, and express a fine and narrow size distribution (^{˜}3.5 nm diameter). The diameter of these nanoparticles was at least two times finer than that of the smallest particles synthesized with the conventional colloidal decomposition techniques (H. B. Na, et al., Angewandte Chemie Int. Ed. 2007, 46, 5397-5401; J. Park, et al., Nature Materials 2004, 3, 891-895; H. L. Si, et al., Crystengcomm 2009, 11, 1128-1132). The nanoparticles were homogeneously distributed in an organic matrix, which means that the dialysis procedure did not completely remove the residual dextran molecules. However, the nanoparticles appear contrasted enough against the matrix, to allow a statistical calculation of the core size distribution using an image analysis software (ImageJ, http://rsbweb.nih.gov/ij/). The calculation was performed over a sample of 594 nanoparticles, and the distribution is presented in FIG. 6. This particle size distribution corresponds to the product shown on the TEM in FIGS. 7a and 7b.

TABLE 1
Nanoparticles synthesized with the single-plasma apparatus and dextran

| | | | | Plasma | Final | | | |
|---|---|---|---|---|---|---|---|---|
| | | Salt | Single or | treatment | concentration | | | |
| Suspension or | | concentration | multiple | time | after dialysis | r₁ | r₂ | |
| solution | Precursor salt | [Gd, Mn], mM | cathode | (min) | [Gd, Mn], mM | mM⁻¹s⁻¹ | mM⁻¹s⁻¹ | r₂/r₁ |
| Mn particles | MnCl₂ | 0.1 | single | 30 | 0.095 | 10.24 | 73.62 | 7.19 |
| Mn particles | MnCl₂ | 5 | single | 30 | 0.113 | 13.96 | 115.2 | 8.25 |
| Mn particles | MnCl₂ | 100 | single | 30 | 0.21 | 8.18 | 65.83 | 8.05 |
| Mn particles | MnCl₂ + ⁶⁴CuCl₂ | 10 | multiple | 5 | 5.58 | 6.83 | 70.63 | 10.34 |
| Mn particles | MnCl₂ + ⁶⁴CuCl₂ | 10 | multiple | 5 | 4.86 | 8.51 | 88.48 | 10.40 |
| Gd particles | GdCl₃ | 0.1 | single | 30 | 0.084 | 16.38 | 20.12 | 1.23 |
| Gd particles | GdCl₃ | 1 | single | 30 | 0.125 | 14.72 | 17.38 | 1.18 |
| Gd particles | GdCl₃ | 10 | single | 30 | 0.305 | 18.75 | 22.54 | 1.20 |
| Gd particles | GdCl₃ | 25 | single | 30 | 0.153 | 18.53 | 23.29 | 1.26 |
| Gd particles | Gd(NO₃)₃ | 0.1 | single | 30 | 0.107 | 14.9 | 17.88 | 1.20 |
| Gd particles | Gd(NO₃)₃ | 1 | single | 30 | 0.125 | 14.97 | 17.82 | 1.19 |
| Gd particles | Gd(NO₃)₃ | 10 | single | 30 | 0.299 | 20.77 | 24.41 | 1.18 |
| Gd particles | Gd(NO₃)₃ | 25 | single | 30 | 0.172 | 14.25 | 17.7 | 1.24 |
| Mn²⁺ in dextran | MnCl₂ + dextran | 0.5 to 10 | — | — | — | 6.81 | 71.89 | 10.56 |
| Gd³⁺ in dextran | Gd(NO₃)₃ + dextran | 0.5 to 10 | — | — | — | 11.084 | 12.707 | 1.15 |
| Gd³⁺ in dextran | GdCl₃ + dextran | 0.5 to 10 | — | — | — | 12.356 | 14.263 | 1.15 |

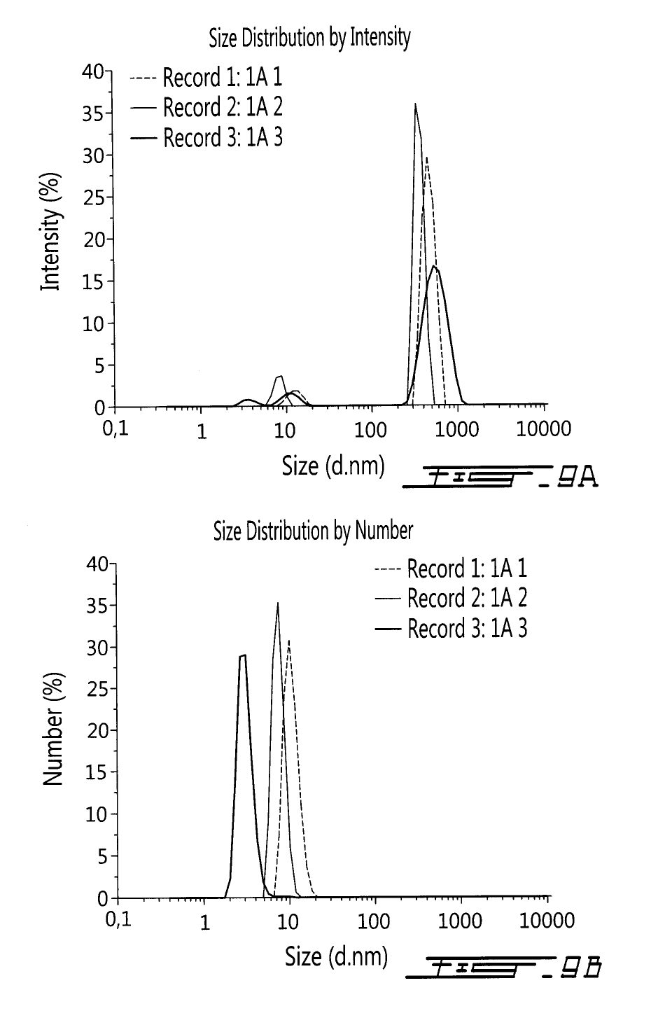

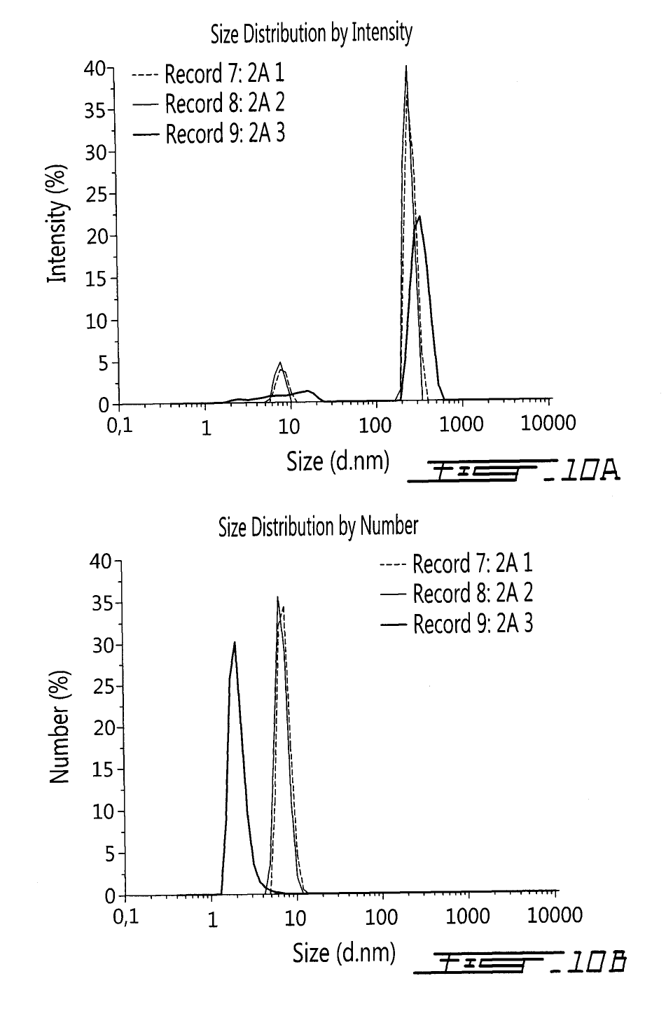

Results from the TEM study were correlated with DLS measurements. First, small hydrodynamic size nanoparticles (<15 nm) were present in all number-weighted spectra as shown in FIGS. 8, 9, and 10. The product synthesized with the single plasma (30-minute treatment) had shown a very high peak for (<15 nm) nanoparticles, both on intensity and number-weighted distributions. FIG. 9 shows that, with a multi-cathode (or multi-plasma) equipment, a certain amount of large-size aggregates of nanoparticles was formed during the process (intensity-weighed graph); however, these peaks disappear on the number-weighted graph, which indicates that very small nanoparticles are dominating in the suspension. FIG. 10 shows confirms the results of FIG. 9 (duplicate sample).

Then, the conversion of this distribution to intensity-weighted spectra, reveals the presence of large size agglomerates (>100 nm) of nanoparticles, or contaminating large-size dextran complexes that were not removed by the initial 0.2 μm filtration procedure. Based on the number-weighted spectra, it is clear that the number of large-size agglomerates was very small. This implies that only a small fraction of all 5000-MW dextran molecules was used in the process. However, this amount was significant enough to be detected in DLS and to affect both the correlogram and the calculated spectra. The presence of large-size agglomerates of nanoparticles appeared on spectra acquired with both single and multi-plasma-synthesized nanoparticles. Because DLS particle size distributions report an indirect measurement of particle size, based on the integration of the correlogram, the presence of a small quantity of large size agglomerate can impact on the positioning of peaks situated at low-hydrodynamic diameters. This explains the variability in the position of peaks corresponding to low-molecular weight species. Therefore, the results from DLS studies are indicative only, and cannot be directly compared with the TEM images and distributions, which give a much more precise assessment of the presence of nanoparticles in dialyzed suspensions.

Size-Distribution Study of GdOₓ Magnetic Metal Nanoparticles:

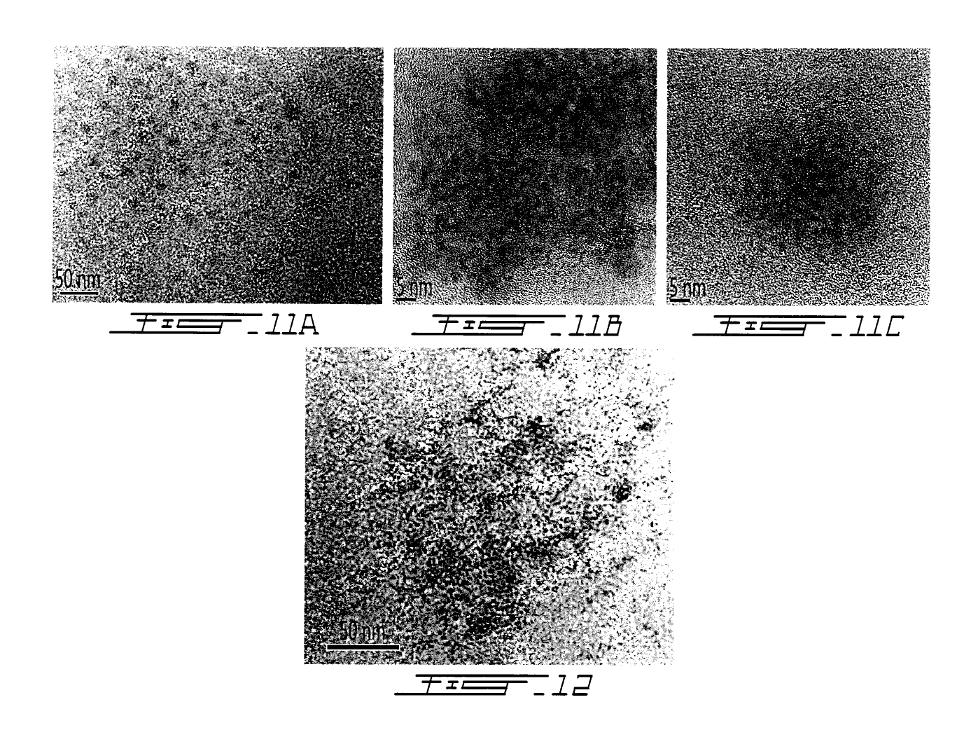

Both gadolinium chloride and nitrate salts were used to synthesize GdOₓ nanocrystals, using the single plasma device and four different precursor concentrations for each salt (0.1, 1, 10 and 25 mM Gd). The more concentrated suspensions were synthesized from 10 mM Gd(NO₃)₃ and 10 mM GdCl₃ solutions. In TEM, the nanoparticles appeared clearly at a magnification of 600 kX as shown in FIG. 11, confirming the very small size of these crystals (5-10 nm diameter). The nanoparticles seemed to aggregate as shown in FIGS. 11b and 11c; as there were evidences of a thick presence of organic materials surrounding the oxide cores, aggregation might occur during the TEM sample preparation. Unfortunately, GdOₓ nanoparticles were poorly contrasted in TEM images; this difficulty was also reported in previous analysis of the polyol-synthesized Gd₂O₃ (M. A. Fortin, et al., Nanotechnology 2007, 18, -; R. Bazzi, et al., Journal of Colloid and Interface Science 2004, 273, 191-197). As a result, it was impossible to generate a statistical particle size distribution with ImageJ particle size analyzer. As for MnOₓ nanoparticles, DLS studies of GdOₓ nanoparticles revealed the presence of large-size agglomerates on intensity-weighted spectra, as well as very small nanoparticles (<15 nm) on number-weighted ones. The visual inspection of all TEM images acquired on nanoparticles synthesized with the nitrate and with the chloride salts, did not reveal significant morphological differences between the products synthesized with both salts.

Size-Distribution Study of FeOₓ Magnetic Metal Nanoparticles:

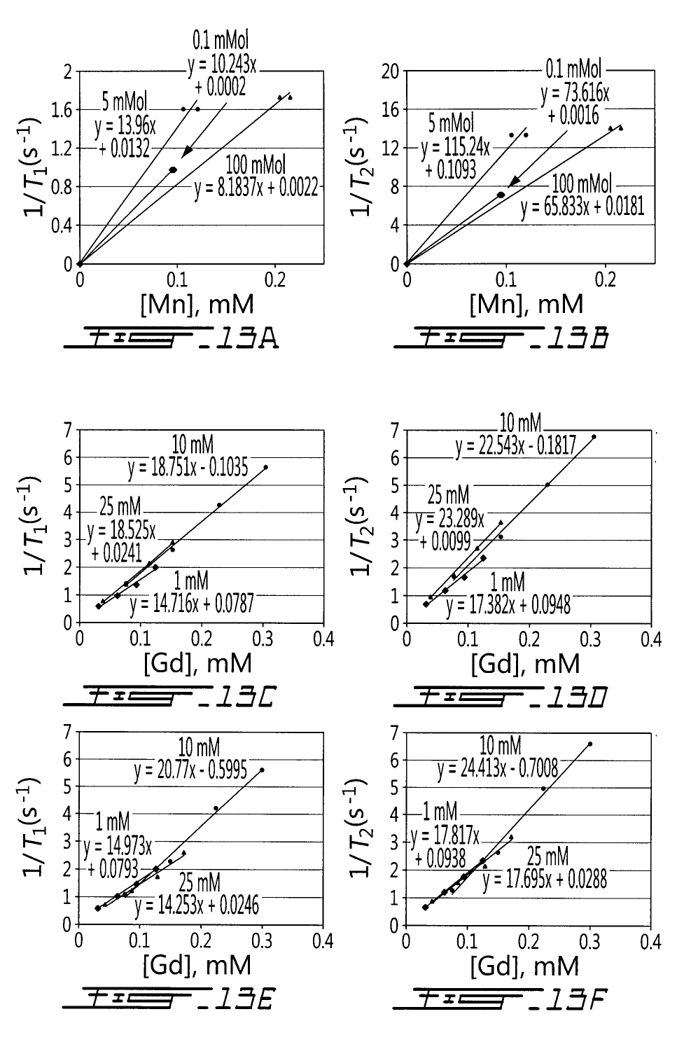

Iron chloride (FeCl₃, 10 mM) was used to synthesize FeOₓ nanocrystals. The iron oxide nanoparticles revealed in TEM as shown in FIG. 12 were ultra small (1-2 nm), as small dots embedded in an organic matrix.

Relaxometric Properties of Dialyzed Suspensions of MnOₓ and GdOₓ Magnetic Metal Nanoparticles:

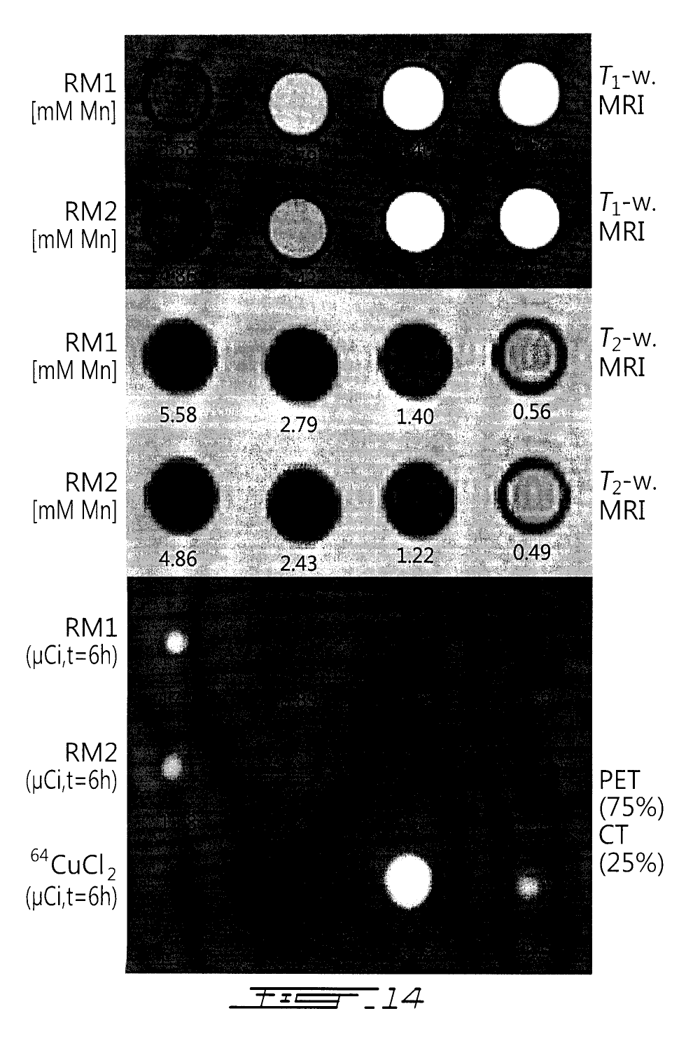

FIG. 13 displays the relaxometric curves of all single-plasma synthesized nanoparticles produced from Mn and Gd ions dissolved in dextran stock solution. More particularly, FIGS. 13 a, c, and e depict longitudinal relaxation rates against the metal concentration in the suspension, whereas FIGS. 13 b, d, and f represent the transversal relaxation rates. All relaxometric measurements were performed after dialysis of the suspensions (21 h, water changed 3 times, 1:1000 volume). The relaxivities (slope of each one of the curves) are presented in Table 1 (above), as well as the measured relaxivities for undialyzed Mn²⁺ and Gd³⁺-containing dextran solutions.

MnOₓ Magnetic Metal Nanoparticle Suspensions Synthesized with the Single-Plasma Apparatus:

The product synthesized using 5 mM precursor solutions provided the highest relaxivities (r₁=13.96 mM⁻¹ s⁻¹ and r₂=115.2 mM⁻¹ s⁻¹). The two other concentrations investigated (0.1 mM and 100 mM) showed somewhat lower relaxivities, although in the same range. The relaxometric ratio (r₂/r₁) provides a good indication of the presence of Mn ions (^{˜}11) or MnO ultra-small nanoparticles (^{˜}8) (H. B. Na, et al., Angewandte Chemie Int. Ed. 2007, 46, 5397-5401; J. Y. Park, et al., European Journal of Inorganic Chemistry 2009, 2477-2481). The relaxometric ratio of all Mn products synthesized with a single-plasma was in the range 7.0-8.5, which indicates a strong presence of nanoparticles compared with Mn²⁺ ions. On the other hand, the suspensions produced with the multi-cathode equipment indicate relaxometric ratios closer to that of ions. In fact, for magnetoradioactive nanoparticles, the dialysis time was kept to 2.5 and 2 hours, which was too short to eliminate all free Mn²⁺ as well as the limited but significant fraction of Mn²⁺ complexed with dextran.

MnO, GdOₓ and FeOₓ Magnetic Metal Nanoparticle Synthesis with the Multi-Cathode Electrochemical Cell, and Polyethylene Glycol and Dimercaptosuccinic Acid as Surfactants:

The synthesis of MnOₓ, GdOₓ and FeOₓ magnetic metal nanoparticles was confirmed by TEM for both surfactants, i.e. PEG and DMSA, as shown in FIG. 15. More particularly, FIGS. 15a and 15b show MnOₓ magnetic metal nanoparticles respectively covered with DMSA and PEG, FIGS. 15c and 15d show GdOₓ magnetic metal nanoparticles respectively covered with DMSA and PEG, and FIGS. 15e and 15f show FeOₓ magnetic metal nanoparticles respectively covered with DMSA and PEG.

As demonstrated by TEM, dispersions of ultra-small magnetic metal nanoparticles are generated using DMSA and PEG-COOH as surfactants, and using Fe, Mn and Gd-containing salts.

⁶⁴Cu-Doped Mn-Based Magnetoradioactive Nanoparticle Suspensions Synthesized with the Multi-Cathode Apparatus:

The concentration of Mn is the highest in the suspensions produced with the multi-cathode device (5.58 and 4.86 mM Mn), about 50 times higher than with the single cathode (or single plasma), and by using only 5 minutes instead of 30 minutes. Thus, the multi-cathode apparatus, which is a multi-plasma apparatus since more than one plasma are generated simultaneously, enhances the efficiency and productivity of the plasma-liquid electrochemical cell.

The proposed method for synthesizing magnetoradioactive nanoparticles and radioactive nanoparticles allows a relatively fast labelling of the nanoparticles with radioisotopes characterized by a relatively short half-life without risks of losing the radioisotopes by dechelation.

Gd-Based Magnetic Metal Nanoparticle Suspensions Synthesized with the Single-Plasma Apparatus and Two Metal Salts:

No significant difference was found between the relaxometric properties of Gd-based nanoparticles synthesized by the nitrate salt, compared with that synthesized with the chloride. In fact, the relaxometric properties were in remarkable agreement. The 10 mM precursor solutions gave both the most concentrated nanoparticle suspensions (0.305 and 0.299 mM Gd after dialysis), as well as the highest relaxivities (r₁=18.75 mM⁻¹ s⁻¹ and 20.77 mM⁻¹ s⁻¹ for GdCl₃ and Gd(NO₃)₃, respectively). These relaxivities were close to two times as high as the relaxivities of untreated aqueous precursor solutions containing dextran and Gd³⁺ ions (11.1-12.4 mM⁻¹ s⁻¹). Suspensions of Gd nanoparticles expressed a r₂/r₁ relaxommetric ratio in the range 1.18-1.26, which corresponds to the data reported in the literature for aqueous suspensions of ultra-small Gd₂O₃ nanoparticles (M. A. Fortin, et al., Nanotechnology 2007, 18, -; M. Ahren, et al., Langmuir 2010, 26, 5753-5762; J. Y. Park, et al., Acs Nano 2009, 3, 3663-3669). Plasma-synthesized Gd-based nanoparticles are therefore a strong “positive” contrast agent for optimal T₁-weighted MR imaging.

Performance of Magnetic and Radioactive Nanoparticles as Dual PET/MRI Contrast Agents:

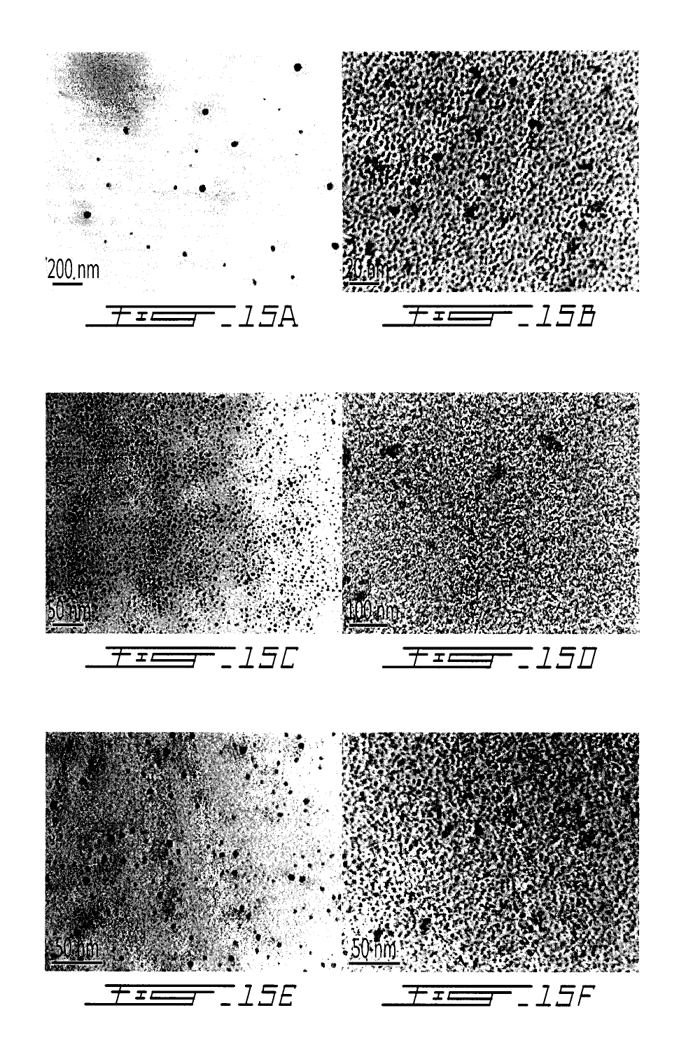

Magnetoradioactive tracers made of ⁶⁴Cu: MnOₓ nanoparticles have been visualized in both MRI and PET/CT. The results of this study are shown in FIG. 14, for both RM1 and RM2 dialyzed products. As expected, the magnetoradioactive products performed remarkably well both as positive and negative contrast agents (in T₁ and T₂-weighted spine echo sequences). In fact, the suspensions were too concentrated to perform optimally as a “positive” contrast agent: as in every aqueous suspension containing a too large amount of Mn ions, here strong T₂ effects quench the signal intensity. As expected for a Mn-containing nanoparticle of r₂/r₁ ratio ranging from 8 to 10, the “positive” signal enhancement effect achieved in T₁ weighted imaging was optimal at a concentration of about 0.5 mM Mn. This indicated that the dialyzed suspension could be diluted in order to achieve optimal relaxivity performance. However, it is worth mentioning again that a certain fraction of this contrast effect is dependent on the presence of contaminating Mn²⁺. A more quantitative contrast-enhancement study is needed in order to eliminate excess Mn²⁺ and therefore their contribution to the signal. The same vials have also been visualized in a dual PET/CT clinical scanner, and these results are also shown in FIG. 14. In FIG. 14, the first four (4) series of dilutions are T₁ and T₂-weighted MRI images demonstrate the performance of the dialysed product as a MRI contrast agent (cross-sections of the tubes). The numerical data under each sample indicate the Mn concentration (in mM Mn). Then, the 5^{th} and second-to-last series of dilutions, are the same samples, images with PET/CT wherein the numerical data under each sample indicate the activity in the tube (μCi).

In order to better delineate the presence of plastic vials in the image, the selected figure is made of the fusion of the PET (75%) and the CT (25%) pictures over the same area. Both RM1 and RM2 feature a significant amount of ⁶⁴Cu in the nanoparticulate suspensions. The highest concentration (100% v./v.) of the magnetoradioactive nanoparticles, corresponding to the undiluted, as-dialyzed products RM1 And RM2, have a mean photon emission that is in the same order as the 13.5 μCi ⁶⁴CuCl₂ standard vial.

Conclusion

GdOₓ, MnOₓ and FeOₓ magnetic metal nanoparticles have been synthesized with single-plasma and multiple-plasma (more particularly, the multi-cathode apparatus) apparatuses. With the multiple plasma apparatus, it has been possible to enhance the production rate and time of synthesis (5 minutes only). The as-synthesized nanoparticles are readily covered with a biocompatible surfactant molecule, and they are dispersed in aqueous media without any ligand-exchange procedure. Magnetic metal nanoparticles are ubiquitous in MRI cellular and molecular tracking procedures. The efficiency of contrast enhancement achieved by using ultra-small nanoparticles is strongly dependent, first on the paramagnetic element used to fabricate the nanoprobes, and then on the fine and narrow particle size distribution.

More particularly, the synthesized nanoparticles have a particle diameter smaller than about 50 nanometers and, in a particular embodiment, smaller than about 25 nanometers. In still another embodiment, the synthesized nanoparticles have a particle diameter smaller than about 15 nanometers. The nanoparticles smaller than about 50 nanometers can be found in agglomerates larger than 100 nanometers but the agglomerates are formed of individual nanoparticles having a particle diameter smaller than about 50 nanometers.

The mechanisms leading to nucleation of metal oxide, metal hydroxide, or metal phosphate nanoparticles are different from the generation of noble metal nanoparticles (e.g. Au, Ag). The generation of metal oxide, metal hydroxide, or metal phosphate nanoparticles by plasma-liquid electrochemistry is not evident based on electrochemical reduction concepts only. In fact, in conventional electrochemistry, the reduction processes of metal ions are mainly guided by the standard reduction potential (Eo) of the corresponding electrochemical half-reactions. In theory, the reduction of any metal expressing a positive Eo should be possible at room temperature, given the presence of a sufficient excess of reducing agent and adequate pH control. This is the case for noble metal nanoparticles plasma synthesis, for which Au and Ag present standard reduction potentials of 1.83 V and 0.7991 V, respectively (see Richmonds et al). Based on electrochemical reduction concepts, the projection of electrons in Ag and Au-containing water, should lead to the nucleation and deposition of Ag and Au.

On the other hand, the synthesis of metal oxide nanoparticles based on the elements such as Mn, Fe, and Gd, and confirmed in the experiments detailed above using transmission electron microscopy (TEM), cannot be explained by direct electrochemical reduction. The mechanism of nucleation and growth certainly involves intermediate nanoparticle nucleation steps. In fact, the standard reduction potentials of Mn, Fe, and Gd are −1.18 V, −0.44V and −2.28 V, respectively. Thus, it is no longer only a matter of injecting electrons from the plasma into the liquid to initiate the reduction reactions. There must be an intermediate step, possibly involving the consumption of —OH (acidification of the solution during plasma treatment was noted when using Mn, Fe, and Gd). Moreover, depending on their energy, radicals can be produced and initiate metal oxide nucleation processes in the liquid. For instance, electrons at a water interface could possibly initiate dissociation reactions and generate a large number of OH— ions through a reaction such as:

2H2O+2e− [symbol] 2OH—+H2.

This mechanism could lead to the synthesis of metal hydroxide nanoparticles.

Therefore, the nucleation of metal oxide nanoparticles by plasma/liquid electrochemistry must be carefully studied in order to identify the basics mechanisms involved, which are certainly very different to that involved in the generation of noble metal nanoparticles (e.g. Au).

One of the main potential applications of plasma-synthesized metal magnetic nanoparticles, is the rapid generation of magnetic metal nanoparticles as contrast agents for magnetic resonance imaging (MRI). In fact, nanoparticles made of the elements Fe, Gd and Mn, are currently used as contrast agents for pre-clinical MRI procedures, but procedures to obtain stable aqueous suspensions implies long (18-48 h) and tedious manipulations. The plasma-liquid electrochemical procedure described above not only produces ultra-small paramagnetic nanoparticles of optimal size for MR imaging, but they also considerably enhance their performance as contrast agents for T₁-weighted imaging.

After dialysis, the suspensions perform remarkably well as magnetic resonance imaging (MRI) contrast agents with high longitudinal relaxivities.

The above-described plasma process can synthesize several types of nanoparticles, which can be used for instance in MRI applications. For instance and without being limitative, ferro-anti-ferrimagnetic, superparamagnetic or paramagnetic material like iron (Fe, Cu), magnetic metal oxide (MeOₓ), magnetic metal phosphate (MePₓO_{y}), magnetic metal hydroxide (Me(OH)ₓ), and mixtures thereof; in all compositions, Me=Gd, Mn, Fe, Dy, Co, Cr, Ni, Ru, Rh, Pd, Or, Ir, Pt, Pb, Sn, Ge, B, Al, Ga, In, Tl, Li, Na, K, Rb, Cs, Fr, Be, Mg, Ca, Sr, Ba, Ra, Cu, Cd, Zn, Sc, Y, La, Ce, Pr, Nd, Sm, Eu, Tb, Ho, Er, Tm, Yb, Lu, Pm. For other uses detailed herein these metals can be radioactive and used for diagnosis or therapy. In all cases, the core can be doped with additional 0.01 to 5.00 mol-% of Gd, Mn,

The electrolyte solution can include between about 0.01 mM and about 1 M of the stabilizer and between about 0.01 mM and about 1 M of the metal ions and, in an alternative embodiment, between about 0.01 mM and about 100 mM of the stabilizer and between about 0.01 mM and about 100 mM of the metal ions, and combination thereof. In a non-limitative embodiment, radioactive atoms can added in a concentration ranging between about 0.001 and about 50 mCi per mL of the electrolyte solution.

Dy, Co, Cr, Ni, Cu, Cd, Zn, F, I, Br, Y, As, In, Ga, La, Ce, Pr, Nd, Sm, Eu, Tb, Ho, Er, Tm, Yb, Lu, Pm. In other words, the synthesized nanoparticles can be doped with one or more radioactive atoms and, more particularly, radioisotope(s) selected from the above group.

The radioactive atoms can be radioactive ions added in the form of a radioactive salt or radioactive molecules.

The synthesized metallic nanoparticles could be non-magnetic and radioactive. For instance and without being limitative, yttrium oxide (or yttria) doped with radioactive yttrium nanoparticles can be synthesized with the above-described processes.

Because of the simplicity and rapidity of this technique, magnetoradioactive nanoparticles with short-lived radioactive atoms have also been synthesized. More particularly, magnetoradioactive nanoparticles were synthesized with a combination of Mn²⁺ and ⁶⁴Cu²⁺ ions. The radioactive atoms are integrated to the metal oxide nanocrystal and not chelated at the surface of the nanoparticles.

For PET applications, the nanoparticles can include, for instance and without being limitative, ¹¹C, ¹³N, ¹⁵O, ^{66/8}Ga, ⁶⁰Cu, ⁵²Fe, ^{61/2/4}Cu, ^{62/3}Zn, ^{70/1/4}As, ^{75/6}Br, ⁸²Rb, ⁸⁶Y, ⁸⁹Zr, ¹¹⁰In, ^{120/4}I, ¹²²Xe, ¹⁸F based radioisotopes and derived tracers.

For SPECT applications, the nanoparticles can include, for instance and without being limitative, contrast enhancing units based on radionucleotides like e.g. ^{99m}Tc, ^{123/5/131}I, ⁶⁷Cu, ⁶⁷Ga, ¹¹¹In, and ²⁰¹Tl.

The synthesized ultra-small suspensions of high-quality magnetoradioactive nanoparticles can be used as magnetoradioactive tracers for dual MRI and PET, for instance.

For therapeutic material, the nanoparticles can include, for instance and without being limitative, UV-C emitting nanoparticles e.g. YPO₄:Pr; metal-containing nanoparticles doped with radionuclides for radiotherapy in the form of metal oxide (MeOₓ), metal hydroxide Me(OH)ₓ, metal phosphate (MePₓO_{y}), doped with radiotherapeutic elements such as (not limited to these) ³²P, ³⁹Sr, ¹⁶⁵Dy, ¹⁰⁹Pd, ¹⁶⁹Er, ¹⁰⁵Rh, ⁷⁷As, ¹⁴⁹Pm, ^{153/7}Sm, ^{212/3}Bi, ^{186/8}Re, ⁶⁷Cu, ⁹⁰Y, ¹³¹I, ^{115m}In, ²¹¹At, ¹⁶⁶Ho, ¹⁹⁹Au, ¹¹¹Ag, ⁴⁷Sc, ^{193m}Pt, ^{117m}Sn, and mixtures thereof.

The “magnetoradioactive” nanoparticles can be used either in MRI procedures, in dual PET/MRI imaging or as radioactive metal nanoparticles for internal radiotherapy. The synthesized nanoparticles being ultra-small, they are thus applicable for most biomedical applications. Biomedical applications include, amongst others, MRI, Positron emission tomography (PET), and Gamma computed tomography (SPECT). Thus, the radioactive or magnetoradioactive nanoparticles can be used as radiotherapeutic agents or radioactive tracers for nuclear image procedures, such as PET/SPECT.

Thus, the magnetoradioactive nanoparticles can be used as contrast agents, MRI tracers, and cell labeling agents, amongst others.

The magnetic metal nanoparticles containing iron oxide(s) can be used as magnetotherapeutic agent or cell labeling agent.

One skilled in the art will appreciate that the nanoparticles comprise a variety of possible compositions, sizes, and shapes.

All documents cited in this text, all documents cited or referenced in documents cited in this text, and any manufacturer's instructions or catalogues for any product cited or mentioned in this text or in any document incorporated into this text, are incorporated herein by reference. Documents incorporated by reference into this text or any teachings therein can be used in the practice of this invention. Documents incorporated by reference into this text are not admitted to be prior art.

Several alternative embodiments and examples have been described and illustrated herein. The embodiments of the invention described above are intended to be exemplary only. A person of ordinary skill in the art would appreciate the features of the individual embodiments, and the possible combinations and variations of the components. A person of ordinary skill in the art would further appreciate that any of the embodiments could be provided in any combination with the other embodiments disclosed herein. It is understood that the invention may be embodied in other specific forms without departing from the spirit or central characteristics thereof. The present examples and embodiments, therefore, are to be considered in all respects as illustrative and not restrictive, and the invention is not to be limited to the details given herein. Accordingly, while the specific embodiments have been illustrated and described, numerous modifications come to mind without significantly departing from the spirit of the invention. The scope of the invention is therefore intended to be limited solely by the scope of the appended claims.

1. A process for synthesizing magnetic metal nanoparticles, the process comprising:
preparing an electrolyte solution including metal ions capable of forming magnetic metal nanoparticles and a stabilizer;
generating, at atmospheric pressure, at least one plasma directed towards an interface of the electrolyte solution; and
recovering, from the electrolyte solution, the synthesized magnetic metal nanoparticles.

2. A process as claimed in claim 1, wherein the metal ions comprise at least one of a ferromagnetic metal and a paramagnetic metal.

3. A process as claimed in claim 2, wherein the at least one of the ferromagnetic metal and the paramagnetic metal comprises at least one of Fe, Co, Ni, Mn, Cr, Gd, Cu, Eu, and Dy.

4. A process as claimed in any one of claims 1 to 3, wherein the synthesized magnetic metal nanoparticles comprise at least one of magnetic metal oxide nanoparticles, magnetic metal phosphate nanoparticles, magnetic metal hydroxide nanoparticles, and mixtures thereof.

5. A process as claimed in any one of claims 1 to 3, wherein the synthesized magnetic metal nanoparticles comprise magnetic metallic nanoparticles selected from the group consisting of: Fe, Cu, and mixtures thereof.

6. A process as claimed in claim 4, wherein the synthesized magnetic metal nanoparticles comprise magnetic metal oxide nanoparticles with a metal selected from the group consisting of: Gd, Mn, Fe, Dy, Co, Cr, Ni, Ru, Rh, Pd, Or, Ir, Pt, Pb, Sn, Ge, B, Al, Ga, In, Tl, Li, Na, K, Rb, Cs, Fr, Be, Mg, Ca, Sr, Ba, Ra, Cu, Cd, Zn, Sc, Y, La, Ce, Pr, Nd, Sm, Eu, Tb, Ho, Er, Tm, Yb, Lu, and Pm.

7. A process as claimed in claim 4, wherein the magnetic metal nanoparticles comprise magnetic metal phosphate nanoparticles with a metal selected from the group consisting of: Gd, Mn, Fe, Dy, Co, Cr, Ni, Ru, Rh, Pd, Or, Ir, Pt, Pb, Sn, Ge, B, Al, Ga, In, Tl, Li, Na, K, Rb, Cs, Fr, Be, Mg, Ca, Sr, Ba, Ra, Cu, Cd, Zn, Sc, Y, La, Ce, Pr, Nd, Sm, Eu, Tb, Ho, Er, Tm, Yb, Lu, and Pm.

8. A process as claimed in claim 4, wherein the magnetic metal nanoparticles comprise magnetic metal hydroxide nanoparticles with a metal selected from the group consisting of: Gd, Mn, Fe, Dy, Co, Cr, Ni, Ru, Rh, Pd, Or, Ir, Pt, Pb, Sn, Ge, B, Al, Ga, In, Tl, Li, Na, K, Rb, Cs, Fr, Be, Mg, Ca, Sr, Ba, Ra, Cu, Cd, Zn, Sc, Y, La, Ce, Pr, Nd, Sm, Eu, Tb, Ho, Er, Tm, Yb, Lu, and Pm.

9. A process as claimed in any one of claims 1 to 8, wherein said generating step further comprises generating simultaneously a plurality of plasma directed towards the interface of the electrolyte solution.

10. A process as claimed in any one of claims 1 to 9, wherein the electrolyte solution is an aqueous electrolyte solution obtained by dissolving a metal ion precursor and the stabilizer in pure water.

11. A process as claimed in claim 10, wherein the metal ion precursor is selected from the group consisting of: metal chlorides, metal nitrates, metal acetates, organometallics, and mixtures thereof.

12. A process as claimed in claim 10, wherein the metal ion precursor is selected from the group consisting of: MnCl₂*4H₂O, Gd(NO₃)₃*6H₂O, GdCl₃*6H₂O, FeCl₃, MnCl₂, Gd(NO₃)₃, GdCl₃, FeCl₃, and mixtures thereof.

13. A process as claimed in any one of claims 1 to 12, wherein the step of generating the at least one plasma is carried out in ambient air.

14. A process as claimed in any one of claims 1 to 13, wherein the stabilizer comprises a surfactant selected from the group consisting of: fructose, dextran, polyethylene glycol, dimercaptosucccinic acid (DMSA), citric acid, and mixtures thereof.

15. A process as claimed in any one of claims 1 to 14, wherein the electrolyte solution comprises between 0.01 mM and 1 M of the stabilizer and between 0.01 mM and 1 M of the metal ions.

16. A process as claimed in any one of claims 1 to 15, wherein the stabilizer is biocompatible.

17. A process as claimed in any one of claims 1 to 16, wherein the stabilizer comprises —OH containing molecules.

18. A process as claimed in any one of claims 1 to 17, wherein the synthesized magnetic metal nanoparticles are covered by the stabilizer in the electrolyte solution.

19. A process as claimed in any one of claims 1 to 18, wherein said generating step comprises injecting at least one of an inert gas and a reactive gas towards the electrolyte solution in at least one gas channel defined in at least one cathode and creating a potential difference between the at least one cathode and at least one anode.

20. A process as claimed in claim 19, wherein the at least one of the inert gas and the reactive gas is selected from the group consisting of: argon, helium, H₂, N₂, NH₃, and mixture thereof.

21. A process as claimed in any one of claims 1 to 20, wherein the synthesized magnetic metal nanoparticles are smaller than about 100 nanometers.

22. A process as claimed in any one of claims 1 to 20, wherein the synthesized magnetic metal nanoparticles are between about 1 nm and about 50 nm.

23. A process as claimed in any one of claims 1 to 20, wherein the synthesized magnetic metal nanoparticles are between about 1 nm and about 15 nm.

24. A process as claimed in any one of claims 1 to 23, further comprising controlling a temperature of the electrolyte solution between about 0° C. and about 50° C.

25. A process as claimed in any one of claims 1 to 24, further comprising controlling a pH of the electrolyte solution between about 5 and about 7.

26. A process as claimed in any one of claims 1 to 25, further comprising adding at least one —OH containing solution to the electrolyte solution during the generating step.

27. A process as claimed in any one of claims 1 to 26, wherein said preparing step further comprises adding at least one radioactive atom to the electrolyte solution.

28. A process as claimed in any one of claims 1 to 27, wherein said generating step further comprises generating at least one micro-plasma.

29. Magnetic metal nanoparticles obtained by the process claimed in any one of claims 1 to 28.

30. Use of magnetic metal nanoparticles obtained by the process claimed in any one of claims 1 to 28 as MRI contrast agent.

31. A contrast agent comprising the magnetic metal nanoparticles obtained by the process claimed in any one of claims 1 to 28.

32. Use of magnetic metal nanoparticles containing at least one iron oxide and obtained by the process claimed in any one of claims 1 to 28 as magnetotherapeutic agent.

33. Use of magnetic metal nanoparticles containing at least one iron oxide and obtained by the process claimed in any one of claims 1 to 28 as cell labeling agent.

34. A process for synthesizing radioactive metal nanoparticles, the process comprising:
preparing an electrolyte solution including metal ions, a stabilizer, and at least one radioactive atom;
generating, at atmospheric pressure, at least one plasma directed towards an interface of the electrolyte solution; and
recovering, from the electrolyte solution, the synthesized radioactive metal nanoparticles with the at least one radioactive atom integrated therein.

35. A process as claimed in claim 34, wherein the at least one radioactive atom is added as at least one of a radioactive salt, a radioactive chloride, a radioactive nitrate, a radioactive acetate, and a radioactive organometallic.

36. A process as claimed in any one of claims 34 and 35, wherein the at least one radioactive atom is at least one of a positron emitter, a beta emitter, an alpha emitter, and a gamma emitter.

37. A process as claimed in any one of claims 34 to 36, wherein the synthesized radioactive metal nanoparticles comprise a core including a crystal containing the at least one radioactive atom.

38. A process as claimed in any one of claims 34 to 37, wherein the at least one radioactive atom comprises at least one radioisotope.

39. A process as claimed in any one of claims 34 to 38, wherein a concentration of the at least one radioactive atom in the synthesized radioactive metal nanoparticles ranges between about 0.01 and about 5.0 mol-%.

40. A process as claimed in any one of claims 34 to 38, wherein the electrolyte solution comprises between about 0.001 and about 50 mCi of the at least one radioactive atom per mL of the electrolyte solution.

41. A process as claimed in any one of claims 34 to 40, wherein the at least one radioactive atom is in a concentration below about 1 wt % in the synthesized radioactive metal nanoparticles.

42. A process as claimed in any one of claims 34 to 41, wherein the at least one radioactive atom is selected from the group consisting of: ¹¹C, ¹³N, ¹⁵O, ^{66/8}Ga, ⁶⁰Cu, ⁵²Fe, ^{61/2/4}Cu, ^{62/3}Zn, ^{70/1/4}As, ^{75/6}Br, ⁸²Rb, ⁸⁶Y, ⁸⁹Zr, ¹¹⁰In, ^{120/4}I, ¹²²Xe, ¹⁸F ^{99m}Tc, ^{123/5/131}I, ⁶⁷Ga, ¹¹¹In, ²⁰¹Tl, ³²P, ³⁹Sr, ¹⁶⁵Dy, ¹⁰⁹Pd, ¹⁶⁹Er, ¹⁰⁵Rh, ⁷⁷As, ¹⁴⁹Pm, ^{153/7}Sm, ¹⁷⁷Lu, ^{212/3}Bi, ^{186/8}Re, ⁶⁷Cu, ⁹⁰Y, ^{115m}In, ²¹¹At, ¹⁶⁶Ho, ¹⁹⁹Au, ¹¹¹Ag, ⁴⁷Sc, ^{193m}Pt, ^{117m}Sn, and mixtures thereof.

43. A process as claimed in any one of claims 34 to 42, wherein the synthesized radioactive metal nanoparticles comprise at least one of metal oxide nanoparticles, metal phosphate nanoparticles, magnetic metal hydroxide nanoparticles, and mixtures thereof.

44. A process as claimed in any one of claims 34 to 42, wherein the synthesized radioactive metal nanoparticles comprise metallic nanoparticles selected from the group consisting of: Fe, Cu, and mixtures thereof.

45. A process as claimed in claim 43, wherein the synthesized radioactive metal nanoparticles comprise metal oxide nanoparticles with a metal selected from the group consisting of: Gd, Mn, Fe, Dy, Co, Cr, Ni, Ru, Rh, Pd, Or, Ir, Pt, Pb, Sn, Ge, B, Al, Ga, In, Tl, Li, Na, K, Rb, Cs, Fr, Be, Mg, Ca, Sr, Ba, Ra, Cu, Cd, Zn, Sc, Y, La, Ce, Pr, Nd, Sm, Eu, Tb, Ho, Er, Tm, Yb, Lu, and Pm.

46. A process as claimed in claim 38, wherein the synthesized radioactive metal nanoparticles comprise metal phosphate nanoparticles with a metal selected from the group consisting of: Gd, Mn, Fe, Dy, Co, Cr, Ni, Ru, Rh, Pd, Or, Ir, Pt, Pb, Sn, Ge, B, Al, Ga, In, Tl, Li, Na, K, Rb, Cs, Fr, Be, Mg, Ca, Sr, Ba, Ra, Cu, Cd, Zn, Sc, Y, La, Ce, Pr, Nd, Sm, Eu, Tb, Ho, Er, Tm, Yb, Lu, and Pm.

47. A process as claimed in claim 38, wherein the synthesized radioactive metal nanoparticles comprise metal hydroxide nanoparticles with a metal selected from the group consisting of: Gd, Mn, Fe, Dy, Co, Cr, Ni, Ru, Rh, Pd, Or, Ir, Pt, Pb, Sn, Ge, B, Al, Ga, In, Tl, Li, Na, K, Rb, Cs, Fr, Be, Mg, Ca, Sr, Ba, Ra, Cu, Cd, Zn, Sc, Y, La, Ce, Pr, Nd, Sm, Eu, Tb, Ho, Er, Tm, Yb, Lu, and Pm.

48. A process as claimed in any one of claims 34 to 42, wherein said generating step further comprises generating simultaneously a plurality of plasma directed towards the interface of the electrolyte solution.

49. A process as claimed in any one of claims 34 to 43, wherein the electrolyte solution is an aqueous electrolyte solution obtained by dissolving a metal ion precursor and the stabilizer in pure water.

50. A process as claimed in claim 44, wherein the metal ion precursor is selected from the group consisting of: metal chlorides, metal nitrates, metal acetates, organometallics, and mixtures thereof.

51. A process as claimed in claim 44, wherein the metal ion precursor is selected from the group consisting of: MnCl₂*4H₂O, Gd(NO₃)₃*6H₂O, GdCl₃*6H₂O, FeCl₃, MnCl₂, Gd(NO₃)₃, GdCl₃, FeCl₃, and mixtures thereof.

52. A process as claimed in any one of claims 34 to 51, wherein the step of generating the at least one plasma is carried out in ambient air.

53. A process as claimed in any one of claims 34 to 52, wherein the stabilizer comprises a surfactant selected from the group consisting of: fructose, dextran, polyethylene glycol, dimercaptosucccinic acid (DMSA), citric acid, and mixtures thereof.

54. A process as claimed in any one of claims 34 to 53, wherein the electrolyte solution comprises between 0.01 mM and 1 M of the stabilizer.

55. A process as claimed in any one of claims 34 to 54, wherein the stabilizer is biocompatible.

56. A process as claimed in any one of claims 34 to 55, wherein the stabilizer comprises —OH containing molecules.

57. A process as claimed in any one of claims 34 to 56, wherein the synthesized radioactive metal nanoparticles are covered by the stabilizer in the electrolyte solution.

58. A process as claimed in any one of claims 34 to 57, wherein said generating step comprises injecting at least one of an inert gas and a reactive gas towards the electrolyte solution in at least one gas channel defined in at least one cathode and creating a potential difference between the at least one cathode and at least one anode.

59. A process as claimed in claim 58, wherein the at least one of the inert gas and the reactive gas is selected from the group consisting of: argon, helium, H₂, N₂, NH₃, and mixtures thereof.

60. A process as claimed in any one of claims 34 to 59, wherein the synthesized radioactive metal nanoparticles are smaller than about 100 nanometers.

61. A process as claimed in any one of claims 34 to 59, wherein the synthesized radioactive metal nanoparticles are between about 1 nm and about 50 nm.

62. A process as claimed in any one of claims 34 to 59, wherein the synthesized radioactive metal nanoparticles are between about 1 nm and about 15 nm.

63. A process as claimed in any one of claims 34 to 62, further comprising controlling a temperature of the electrolyte solution between about 0° C. and about 50° C.

64. A process as claimed in any one of claims 34 to 63, further comprising controlling a pH of the electrolyte solution between about 5 and about 7.

65. A process as claimed in any one of claims 34 to 64, further comprising adding at least one —OH containing solution to the electrolyte solution during the generating step.

66. A process as claimed in any one of claims 34 to 65, wherein said generating step further comprises generating at least one micro-plasma.

67. Radioactive metal nanoparticles obtained by the process claimed in any one of claims 34 to 66.

68. A radiotherapeutic agent comprising the radioactive metal nanoparticles obtained by the process claimed in any one of claims 34 to 66.

69. A radioactive tracer for nuclear image procedures comprising the radioactive metal nanoparticles obtained by the process claimed in any one of claims 34 to 66.

70. A process for synthesizing magnetoradioactive metal nanoparticles, the process comprising:
preparing an electrolyte solution including metal ions capable of forming magnetic metal nanoparticles, a stabilizer, and at least one radioactive atom;
generating, at atmospheric pressure, at least one plasma directed towards an interface of the electrolyte solution; and
recovering, from the electrolyte solution, the synthesized magnetoradioactive metal nanoparticles.

71. A process as claimed in claim 70, wherein the metal ions comprise at least one of a ferromagnetic metal and a paramagnetic metal.

72. A process as claimed in claim 71, wherein the at least one of the ferromagnetic metal and the paramagnetic metal comprises at least one of Fe, Co, Ni, Mn, Cr, Cu, Gd, Eu, and Dy.

73. A process as claimed in any one of claims 70 to 72, wherein the synthesized magnetoradioactive metal nanoparticles comprise at least one of magnetic metal oxide nanoparticles, magnetic metal phosphate nanoparticles, magnetic metal hydroxide nanoparticles, and mixtures thereof.

74. A process as claimed in any one of claims 70 to 72, wherein the synthesized magnetoradioactive metal nanoparticles comprise magnetic metallic nanoparticles selected from the group consisting of: Fe, Cu, and mixtures thereof.

75. A process as claimed in claim 74, wherein the synthesized magnetoradioactive metal nanoparticles comprise magnetic metal oxide nanoparticles with a metal selected from the group consisting of: Gd, Mn, Fe, Dy, Co, Cr, Ni, Ru, Rh, Pd, Or, Ir, Pt, Pb, Sn, Ge, B, Al, Ga, In, Tl, Li, Na, K, Rb, Cs, Fr, Be, Mg, Ca, Sr, Ba, Ra, Cu, Cd, Zn, Sc, Y, La, Ce, Pr, Nd, Sm, Eu, Tb, Ho, Er, Tm, Yb, Lu, and Pm.

76. A process as claimed in claim 74, wherein the synthesized magnetoradioactive metal nanoparticles comprise magnetic metal phosphate nanoparticles with a metal selected from the group consisting of: Gd, Mn, Fe, Dy, Co, Cr, Ni, Ru, Rh, Pd, Or, Ir, Pt, Pb, Sn, Ge, B, Al, Ga, In, Tl, Li, Na, K, Rb, Cs, Fr, Be, Mg, Ca, Sr, Ba, Ra, Cu, Cd, Zn, Sc, Y, La, Ce, Pr, Nd, Sm, Eu, Tb, Ho, Er, Tm, Yb, Lu, and Pm.

77. A process as claimed in claim 74, wherein the synthesized magnetoradioactive metal nanoparticles comprise magnetic metal hydroxide nanoparticles with a metal selected from the group consisting of: Gd, Mn, Fe, Dy, Co, Cr, Ni, Ru, Rh, Pd, Or, Ir, Pt, Pb, Sn, Ge, B, Al, Ga, In, Tl, Li, Na, K, Rb, Cs, Fr, Be, Mg, Ca, Sr, Ba, Ra, Cu, Cd, Zn, Sc, Y, La, Ce, Pr, Nd, Sm, Eu, Tb, Ho, Er, Tm, Yb, Lu, and Pm.

78. A process as claimed in any one of claims 70 to 77, wherein the at least one radioactive atom is added as at least one of a radioactive salt, a radioactive chloride, a radioactive nitrate, a radioactive acetate, and a radioactive organometallic.

79. A process as claimed in any one of claims 70 to 78, wherein the at least one radioactive atom is at least one of a positron emitter, a beta emitter, an alpha emitter, and a gamma emitter.

80. A process as claimed in any one of claims 70 to 79, wherein the synthesized radioactive metal nanoparticles comprise the at least one radioactive atom integrated to the synthesized radioactive metal nanoparticles.

81. A process as claimed in any one of claims 70 to 80, wherein the synthesized radioactive metal nanoparticles comprise a core including a crystal containing the at least one radioactive atom.

82. A process as claimed in any one of claims 70 to 81, wherein the at least one radioactive atom comprises at least one radioisotope.

83. A process as claimed in any one of claims 70 to 82, wherein a concentration of the at least one radioactive atom in the synthesized radioactive metal nanoparticles ranges between about 0.01 and about 5.0 mol-%.

84. A process as claimed in any one of claims 70 to 82, wherein the electrolyte solution comprises between about 0.001 and about 50 mCi of the at least one radioactive atom per mL of the electrolyte solution.

85. A process as claimed in any one of claims 70 to 84, wherein the at least one radioactive atom is in a concentration below about 1 wt % in the synthesized radioactive metal nanoparticles.

86. A process as claimed in any one of claims 70 to 85, wherein the at least one radioactive atom is selected from the group consisting of: ¹¹C, ¹³N, ¹⁵O, ^{66/8}Ga, ⁶⁰Cu, ⁵²Fe, ^{61/2/4}Cu, ^{62/3}Zn, ^{70/1/4}As, ^{75/6}Br, ⁸²Rb, ⁸⁶Y, ⁸⁹Zr, ¹¹⁰In, ^{120/4}I, ¹²²Xe, ¹⁸F ^{99m}Tc, ^{123/5/131}I, ⁶⁷Ga, ¹¹¹In, ²⁰¹Tl, ³²P, ³⁹Sr, ¹⁶⁵Dy, ¹⁰⁹Pd, ¹⁶⁹Er, ¹⁰⁵Rh, ⁷⁷As, ¹⁴⁹Pm, ^{153/7}Sm, ¹⁷⁷Lu, ^{212/3}Bi, ^{186/8}Re, ⁶⁷Cu, ⁹⁰Y, ^{115m}In, ²¹¹At, ¹⁶⁶Ho, ¹⁹⁹Au, ¹¹¹Ag, ⁴⁷Sc, ^{193m}Pt, ^{117m}Sn, and mixtures thereof.

87. A process as claimed in any one of claims 70 to 86, wherein said generating step further comprises generating simultaneously a plurality of plasma directed towards the interface of the electrolyte solution.

88. A process as claimed in any one of claims 70 to 87, wherein the electrolyte solution is an aqueous electrolyte solution obtained by dissolving a metal ion precursor and the stabilizer in pure water.

89. A process as claimed in claim 88, wherein the metal ion precursor is selected from the group consisting of: metal chlorides, metal nitrates, metal acetates, organometallics, and mixtures thereof.

90. A process as claimed in claim 88, wherein the metal ion precursor is selected from the group consisting of: MnCl₂*4H₂O, Gd(NO₃)₃*6H₂O, GdCl₃*6H₂O, FeCl₃, MnCl₂, Gd(NO₃)₃, GdCl₃, FeCl₃, and mixtures thereof.

91. A process as claimed in any one of claims 70 to 90, wherein the step of generating the at least one plasma is carried out in ambient air.

92. A process as claimed in any one of claims 70 to 91, wherein the stabilizer comprises a surfactant selected from the group consisting of: fructose, dextran, polyethylene glycol, dimercaptosucccinic acid (DMSA), citric acid, and mixtures thereof.

93. A process as claimed in any one of claims 70 to 92, wherein the electrolyte solution comprises between 0.01 mM and 1 M of the stabilizer and between 0.01 mM and 1 M of the metal ions.

94. A process as claimed in any one of claims 70 to 93, wherein the stabilizer is biocompatible.

95. A process as claimed in any one of claims 70 to 94, wherein the stabilizer comprises —OH containing molecules.

96. A process as claimed in any one of claims 70 to 95, wherein the synthesized magnetoradioactive metal nanoparticles are covered by the stabilizer in the electrolyte solution.

97. A process as claimed in any one of claims 70 to 96, wherein said generating step comprises injecting at least one of an inert gas and a reactive gas towards the electrolyte solution in at least one gas channel defined in at least one cathode and creating a potential difference between the at least one cathode and at least one anode.

98. A process as claimed in claim 97, wherein the at least one of the inert gas and the reactive gas is selected from the group consisting of: argon, helium, H₂, N₂, NH₃, and mixtures thereof.

99. A process as claimed in any one of claims 70 to 98, wherein the synthesized magnetoradioactive metal nanoparticles are smaller than about 100 nanometers.

100. A process as claimed in any one of claims 70 to 98, wherein the synthesized magnetoradioactive metal nanoparticles are between about 1 and about 50 nm.

101. A process as claimed in any one of claims 70 to 98, wherein the synthesized magnetoradioactive metal nanoparticles are between about 1 and about 15 nm.

102. A process as claimed in any one of claims 70 to 101, further comprising controlling a temperature of the electrolyte solution between about 0° C. and about 50° C.

103. A process as claimed in any one of claims 70 to 102, further comprising controlling a pH of the electrolyte solution between about 5 and about 7.

104. A process as claimed in any one of claims 70 to 103, further comprising adding at least one —OH containing solution to the electrolyte solution during the generating step.

105. A process as claimed in any one of claims 70 to 104, wherein said generating step further comprises generating at least one micro-plasma.

106. Magnetoradioactive metal nanoparticles obtained by the process claimed in any one of claims 70 to 105.

107. A PET tracer comprising the magnetoradioactive metal nanoparticles obtained by the process claimed in any one of claims 70 to 105.

108. Use of magnetoradioactive metal nanoparticles claimed in any one of claims 70 to 105 for dual PET/MRI.

109. A contrast agent comprising the magnetoradioactive metal nanoparticles obtained by the process claimed in any one of claims 70 to 105.

110. A MRI tracer comprising the magnetoradioactive metal nanoparticles obtained by the process claimed in any one of claims 70 to 105.

111. A cell labeling agent comprising the magnetoradioactive metal nanoparticles obtained by the process claimed in any one of claims 70 to 105.

112. A magnetotherapeutic agent comprising the magnetoradioactive metal nanoparticles obtained by the process claimed in any one of claims 70 to 105.

113. Radioactive metal nanoparticles synthesized by a plasma-liquid electrochemical process at atmospheric pressure and comprising a metal element content and at least one radioactive atom integrated in a crystal of the synthesized radioactive metal nanoparticles.

114. Radioactive metal nanoparticles as claimed in claim 113, wherein the synthesized radioactive metal nanoparticles comprise at least one of magnetic metal oxide nanoparticles, magnetic metal phosphate nanoparticles, magnetic metal hydroxide nanoparticles, and mixtures thereof.

115. Radioactive metal nanoparticles as claimed in claim 114, wherein the synthesized radioactive metal nanoparticles comprise metal oxide nanoparticles with a metal selected from the group consisting of: Gd, Mn, Fe, Dy, Co, Cr, Ni, Ru, Rh, Pd, Or, Ir, Pt, Pb, Sn, Ge, B, Al, Ga, In, Tl, Li, Na, K, Rb, Cs, Fr, Be, Mg, Ca, Sr, Ba, Ra, Cu, Cd, Zn, Sc, Y, La, Ce, Pr, Nd, Sm, Eu, Tb, Ho, Er, Tm, Yb, Lu, and Pm.

116. Radioactive metal nanoparticles as claimed in claim 114, wherein the synthesized radioactive metal nanoparticles comprise magnetic metal phosphate nanoparticles with a metal selected from the group consisting of: Gd, Mn, Fe, Dy, Co, Cr, Ni, Ru, Rh, Pd, Or, Ir, Pt, Pb, Sn, Ge, B, Al, Ga, In, Tl, Li, Na, K, Rb, Cs, Fr, Be, Mg, Ca, Sr, Ba, Ra, Cu, Cd, Zn, Sc, Y, La, Ce, Pr, Nd, Sm, Eu, Tb, Ho, Er, Tm, Yb, Lu, and Pm.

117. Radioactive metal nanoparticles as claimed in claim 114, wherein the synthesized radioactive metal nanoparticles comprise magnetic metal hydroxide nanoparticles with a metal selected from the group consisting of: Gd, Mn, Fe, Dy, Co, Cr, Ni, Ru, Rh, Pd, Or, Ir, Pt, Pb, Sn, Ge, B, Al, Ga, In, Tl, Li, Na, K, Rb, Cs, Fr, Be, Mg, Ca, Sr, Ba, Ra, Cu, Cd, Zn, Sc, Y, La, Ce, Pr, Nd, Sm, Eu, Tb, Ho, Er, Tm, Yb, Lu, and Pm.

118. Radioactive metal nanoparticles as claimed in any one of claims 113 to 117, wherein the at least one radioactive atom comprises at least one of a positron emitter, a beta emitter, an alpha emitter, and a gamma emitter.

119. Radioactive metal nanoparticles as claimed in any one of claims 113 to 118, further comprising a core including the crystal containing the at least one radioactive atom.

120. Radioactive metal nanoparticles as claimed in any one of claims 113 to 119, wherein a concentration of the at least one radioactive atom in the synthesized radioactive metal nanoparticles ranges between about 0.01 and about 5.0 mol-%.

121. Radioactive metal nanoparticles as claimed in any one of claims 113 to 119, wherein the at least one radioactive atom is in a concentration below about 1 wt % in the synthesized radioactive metal nanoparticles.

122. Radioactive metal nanoparticles as claimed in any one of claims 113 to 120, wherein the at least one radioactive atom is selected from the group consisting of: ¹¹C, ¹³N, ¹⁵O, ^{66/8}Ga, ⁶⁰Cu, ⁵²Fe, ^{61/2/4}Cu, ^{62/3}Zn, ^{70/1/4}As, ^{75/6}Br, ⁸²Rb, ⁸⁶Y, ⁸⁹Zr, ¹¹⁰In, ^{120/4}I, ¹²²Xe, ¹⁸F ^{99m}Tc, ^{123/5/131}I, ⁶⁷Ga, ¹¹¹In, ²⁰¹Tl, ³²P, ³⁹Sr, ¹⁶⁵Dy, ¹⁰⁹Pd, ¹⁶⁹Er, ¹⁰⁵Rh, ⁷⁷As, ¹⁴⁹Pm, ^{153/7}Sm, ¹⁷⁷Lu, ^{212/3}Bi, ^{186/8}Re, ⁶⁷Cu, ⁹⁰Y, ^{115m}In, ²¹¹At, ¹⁶⁶Ho, ¹⁹⁹Au, ¹¹¹Ag, ⁴⁷Sc, ^{193m}Pt, ^{117m}Sn, and mixtures thereof.

123. Radioactive metal nanoparticles as claimed in any one of claims 113 to 121, wherein the synthesized radioactive metal nanoparticles are covered by a stabilizer.

124. Radioactive metal nanoparticles as claimed in claim 123, wherein the stabilizer comprises a surfactant selected from the group consisting of: fructose, dextran, polyethylene glycol, dimercaptosucccinic acid (DMSA), citric acid, and mixtures thereof.

125. Radioactive metal nanoparticles as claimed in claim 123, wherein the stabilizer is biocompatible.

126. Radioactive metal nanoparticles as claimed in any one of claims 113 to 125, wherein the synthesized radioactive metal nanoparticles are smaller than about 100 nanometers.

127. Radioactive metal nanoparticles as claimed in any one of claims 113 to 125, wherein the synthesized radioactive metal nanoparticles are between about 1 and about 50 nm.

128. Radioactive metal nanoparticles as claimed in any one of claims 113 to 125, wherein the synthesized radioactive metal nanoparticles are between about 1 and about 15 nm.

129. Magnetoradioactive metal nanoparticles synthesized by a plasma-liquid electrochemical process at atmospheric pressure and comprising a metal element content and at least one radioactive atom integrated in a crystal of the synthesized magnetoradioactive metal nanoparticles.

130. Magnetoradioactive metal nanoparticles as claimed in claim 129, wherein the metal element content comprises at least one of a ferromagnetic metal and a paramagnetic metal.

131. Magnetoradioactive metal nanoparticles as claimed in claim 130, wherein the at least one of the ferromagnetic metal and the paramagnetic metal comprises at least one of Fe, Co, Ni, Mn, Cr, Cu, Eu, Gd, and Dy.

132. Magnetoradioactive metal nanoparticles as claimed in any one of claims 129 to 131, wherein the synthesized magnetoradioactive metal nanoparticles comprise at least one of magnetic metal oxide nanoparticles, magnetic metal phosphate nanoparticles, magnetic metal hydroxide nanoparticles, and mixtures thereof.

133. Magnetoradioactive metal nanoparticles as claimed in any one of claims 129 to 131, wherein the synthesized magnetoradioactive metal nanoparticles comprise magnetic metallic nanoparticles selected from the group consisting of: Fe, Cu, and mixtures thereof.

134. Magnetoradioactive metal nanoparticles as claimed in claim 132, wherein the synthesized magnetoradioactive metal nanoparticles comprise magnetic metal oxide nanoparticles with a metal selected from the group consisting of: Gd, Mn, Fe, Dy, Co, Cr, Ni, Ru, Rh, Pd, Or, Ir, Pt, Pb, Sn, Ge, B, Al, Ga, In, Tl, Li, Na, K, Rb, Cs, Fr, Be, Mg, Ca, Sr, Ba, Ra, Cu, Cd, Zn, Sc, Y, La, Ce, Pr, Nd, Sm, Eu, Tb, Ho, Er, Tm, Yb, Lu, and Pm.

135. Magnetoradioactive metal nanoparticles as claimed in claim 132, wherein the synthesized magnetoradioactive metal nanoparticles comprise magnetic metal phosphate nanoparticles with a metal selected from the group consisting of: Gd, Mn, Fe, Dy, Co, Cr, Ni, Ru, Rh, Pd, Or, Ir, Pt, Pb, Sn, Ge, B, Al, Ga, In, Tl, Li, Na, K, Rb, Cs, Fr, Be, Mg, Ca, Sr, Ba, Ra, Cu, Cd, Zn, Sc, Y, La, Ce, Pr, Nd, Sm, Eu, Tb, Ho, Er, Tm, Yb, Lu, and Pm.

136. Magnetoradioactive metal nanoparticles as claimed in claim 132, wherein the synthesized magnetoradioactive metal nanoparticles comprise magnetic metal hydroxide nanoparticles with a metal selected from the group consisting of: Gd, Mn, Fe, Dy, Co, Cr, Ni, Ru, Rh, Pd, Or, Ir, Pt, Pb, Sn, Ge, B, Al, Ga, In, Tl, Li, Na, K, Rb, Cs, Fr, Be, Mg, Ca, Sr, Ba, Ra, Cu, Cd, Zn, Sc, Y, La, Ce, Pr, Nd, Sm, Eu, Tb, Ho, Er, Tm, Yb, Lu, and Pm.

137. Magnetoradioactive metal nanoparticles as claimed in any one of claims 129 to 136, wherein the at least one radioactive atom comprises at least one of a positron emitter, a beta emitter, an alpha emitter, and a gamma emitter.

138. Magnetoradioactive metal nanoparticles as claimed in any one of claims 129 to 137, wherein the at least one radioactive atom is integrated to the synthesized magnetoradioactive metal nanoparticles.

139. Magnetoradioactive metal nanoparticles as claimed in any one of claims 129 to 138, further comprising a core including a crystal containing the at least one radioactive atom.

140. Magnetoradioactive metal nanoparticles as claimed in any one of claims 129 to 139, wherein a concentration of the at least one radioactive atom in the synthesized radioactive metal nanoparticles ranges between about 0.01 and about 5.0 mol-%.

141. Magnetoradioactive metal nanoparticles as claimed in any one of claims 129 to 139, wherein the at least one radioactive atom is in a concentration below about 1 wt % in the synthesized magnetoradioactive metal nanoparticles.

142. Magnetoradioactive metal nanoparticles as claimed in any one of claims 129 to 141, wherein the at least one radioactive atom is selected from the group consisting of: ¹¹C, ¹³N, ¹⁵O, ^{66/8}Ga, ⁶⁰Cu, ⁵²Fe, ^{61/2/4}Cu, ^{62/3}Zn, ^{70/1/4}As, ^{75/6}Br, ⁸²Rb, ⁸⁶Y, ⁸⁹Zr, ¹¹⁰In, ^{120/4}I, ¹²²Xe, ¹⁸F ^{99m}Tc, ^{123/5/131}I, ⁶⁷Ga, ¹¹¹In, ²⁰¹Tl, ³²P, ³⁹Sr, ¹⁶⁵Dy, ¹⁰⁹Pd, ¹⁶⁹Er, ¹⁰⁵Rh, ⁷⁷As, ¹⁴⁹Pm, ^{153/7}Sm, ¹⁷⁷Lu, ^{212/3}Bi, ^{186/8}Re, ⁶⁷Cu, ⁹⁰Y, ^{115m}In, ²¹¹At, ¹⁶⁶Ho, ¹⁹⁹Au, ¹¹¹Ag, ⁴⁷Sc, ^{193m}Pt, ^{117m}Sn, and mixtures thereof.

143. Magnetoradioactive metal nanoparticles as claimed in any one of claims 129 to 142, wherein the synthesized magnetoradioactive metal nanoparticles are covered by a stabilizer.

144. Magnetoradioactive metal nanoparticles as claimed in claim 143, wherein the stabilizer comprises a surfactant selected from the group consisting of: fructose, dextran, polyethylene glycol, dimercaptosucccinic acid (DMSA), citric acid, and mixtures thereof.

145. Magnetoradioactive metal nanoparticles as claimed in claim 143, wherein the stabilizer is biocompatible.

146. Magnetoradioactive metal nanoparticles as claimed in any one of claims 129 to 145, wherein the synthesized magnetoradioactive metal nanoparticles are smaller than about 100 nanometers.

147. Magnetoradioactive metal nanoparticles as claimed in any one of claims 129 to 145, wherein the synthesized magnetoradioactive metal nanoparticles are between about 1 and about 50 nm.

148. Magnetoradioactive metal nanoparticles as claimed in any one of claims 129 to 145, wherein the synthesized magnetoradioactive metal nanoparticles are between about 1 and about 15 nm.

149. A multi-plasma apparatus for the synthesis of nanoparticles comprising:
an electrolyte solution container;
at least one cathode defining a plurality of spaced-apart gas channels having a gas output directed towards the electrolyte solution container;
at least one anode electrically connectable to the at least one cathode;
a gas supply connectable to the plurality of spaced-apart gas channels of the at least one cathode; and
a power supply connectable to the at least one cathode and the at least one anode for creating a potential difference therebetween.

150. A multi-plasma apparatus as claimed in claim 149, wherein a plasma is generated in each one of the gas channels when gas flows therein and a potential difference between the at least one anode and the at least one cathode is created.

151. A multi-plasma apparatus as claimed in one of claims 149 and 150, wherein the at least one cathode comprises a cathode body with a plurality of through holes in gas communication with the gas supply and defining at least a section of the gas channels.

152. A multi-plasma apparatus as claimed in claim 151, wherein the at least one anode comprises an anode body with a plurality of through holes in gas communication with the through holes defined in the cathode body.

153. A multi-plasma apparatus as claimed in claim 152, further comprising an insulating layer extending between the cathode body and the anode body and comprising a plurality of through holes in gas communication with the through holes defined in the anode body and the cathode body.

154. A multi-plasma apparatus as claimed in claim 153, wherein the cathode body, the insulating layer, and the anode body are superposed and the through holes defined in the cathode body, the anode body, and the insulating layer define the gas channels.

155. A multi-plasma apparatus as claimed in claim 149, wherein the at least one cathode comprises a plurality of spaced-apart cathode capillary tubes spaced apart from one another and from the at least one anode and each one of the cathode capillary tubes has a tip pointing towards the electrolyte solution container, each one of the cathode capillary tubes has one of the gas channels defined therein.

156. A multi-plasma apparatus as claimed in claim 155, wherein the cathode capillary tubes comprise stainless steel and the at least one anode comprises graphite.

157. A multi-plasma apparatus as claimed in one of claims 155 and 156, wherein the cathode capillary tubes are disposed around the at least one anode.

158. A multi-plasma apparatus as claimed in any one of claims 155 to 157, wherein the electrolyte solution container contains an aqueous electrolyte solution and the tips of the cathode capillary tubes are substantially equally spaced-apart from an interface of the aqueous electrolyte solution with air.

159. A multi-plasma apparatus as claimed in claim 158, wherein the tips of the cathode capillary tubes are spaced apart from the interface at least about 1 mm.

160. A multi-plasma apparatus as claimed in any one of claims 149 to 159, wherein the gas channels are substantially equally spaced-apart from the adjacent gas channels.

161. A multi-plasma apparatus as claimed in any one of claims 149 to 160, wherein the gas channels extend substantially parallel to one another.

162. A multi-plasma apparatus as claimed in any one of claims 149 to 161, wherein the electrolyte solution container contains an aqueous electrolyte solution.

163. A multi-plasma apparatus as claimed in any one of claims 149 to 162, wherein the at least one anode is at least partially immersable in an electrolyte solution contained in the electrolyte solution container.

164. A multi-plasma apparatus as claimed in any one of claims 149 to 162, wherein the at least one anode is mounted outwardly of an electrolyte solution contained in the electrolyte solution container.

165. A multi-plasma apparatus as claimed in any one of claims 149 to 164, further comprising at least one of a thermoelectric cooling device to allow for in situ temperature control and a pH probe linked to an automated pH adjustment system.

166. A multi-plasma apparatus as claimed in any one of claims 149 to 165, wherein the apparatus operates at atmospheric pressure.

167. A multi-plasma apparatus as claimed in any one of claims 149 to 166, wherein gas supply comprises at least one of an inert gas supply and a reactive gas supply.

168. A multi-plasma apparatus as claimed in any one of claims 149 to 167, wherein said multi-plasma apparatus generates a plurality of micro-plasmas.

169. A method for synthesizing nanoparticles, the method comprising:
providing at least one cathode defining a plurality of spaced-apart gas channels having a gas output directed towards an electrolyte solution contained in an electrolyte solution container;
providing at least one anode electrically connected to the at least one cathode;
supplying gas into the gas channels towards the electrolyte solution; and
creating a potential difference between the at least one cathode and the at least one anode sufficient to ignite simultaneously plasmas in the gas channels.

170. A method as claimed in claim 169, wherein the at least one cathode comprises a cathode body with a plurality of through holes defining at least a first section of the gas channels and in which the gas is supplied.

171. A method as claimed in claim 170, wherein the at least one anode comprises an anode body with a plurality of through holes in gas communication with the through holes defined in the cathode body and defining a second section of the gas channels.

172. A method as claimed in claim 171, further comprising providing an insulating layer extending between the cathode body and the anode body and comprising a plurality of through holes in gas communication with the through holes defined in the anode body and the cathode body and defining a third section of the gas channels and wherein the gas flows continuously in the first section, the second section, and the third section of the gas channels.

173. A method as claimed in claim 169, wherein the at least one cathode comprises a plurality of spaced-apart cathode capillary tubes spaced apart from one another and from the at least one anode and each one of the cathode capillary tubes has a tip pointing towards the electrolyte solution, each one of the cathode capillary tubes has one of the gas channels defined therein.

174. A method as claimed in claim 173, wherein the cathode capillary tubes comprise stainless steel and the at least one anode comprises graphite.

175. A method as claimed in any one of claims 169 to 174, wherein the electrolyte solution contained in the electrolyte solution container is an aqueous electrolyte solution.

176. A method as claimed in any one of claims 169 to 175, wherein the gas channels are substantially equally spaced-apart from the adjacent gas channels.

177. A method as claimed in any one of claims 169 to 176, wherein the gas channels extend substantially parallel to one another.

178. A method as claimed in any one of claims 169 to 177, further comprising at least partially immersed the at least one anode in the electrolyte solution contained in the electrolyte solution container.

179. A method as claimed in any one of claims 169 to 178, wherein the at least one anode is mounted outwardly of an electrolyte solution contained in the electrolyte solution container.

180. A method as claimed in any one of claims 169 to 179, further comprising cooling the electrolyte solution when plasmas are ignited.

181. A method as claimed in any one of claims 169 to 180, further controlling a temperature of the electrolyte solution when plasmas are ignited.

182. A method as claimed in any one of claims 169 to 181, further comprising increasing an alkalinity of the electrolyte solution when plasmas are ignited.

183. A method as claimed in any one of claims 169 to 182, further controlling a pH of the electrolyte solution when plasmas are ignited.

184. A method as claimed in any one of claims 169 to 183, wherein the plasmas are ignited at atmospheric pressure and ambient air.

185. A method as claimed in any one of claims 169 to 184, wherein the supplying step comprises supplying at least one of an inert gas and a reactive gas.

186. A method as claimed in any one of claims 169 to 185, wherein a plurality of micro-plasmas are ignited simultaneously.
